(19) United States
(12) Patent Application Publication
(10) Pub. No.: US 2009/0016997 A1
(43) Pub. Date: Jan. 15, 2009

(54) Autologous/allogeneic human DNA grafting, anti-and reverse aging stem cell, and bone marrow compositions/methods

(76) Inventors: Alecia A. Hathaway, Fort Worth, TX (US); William C. Orr, Denver, CO (US)

Correspondence Address: HOGAN & HARTSON LLP, ONE TABOR CENTER, SUITE 1500, 1200 SEVENTEENTH ST, DENVER, CO, 80202, US

(21) Appl. No.: 12/082,669

(22) Filed: Apr. 11, 2008

Related U.S. Application Data
(60) Provisional application No. 60/923,145, filed on Apr. 11, 2007.

(51) Int. Cl. A61K 35/12; A61K 35/28

(52) U.S. Cl. 424/93.7; 424/577

(57) ABSTRACT

Anti-aging compositions and methods for delivering (engrafting) younger compatible human DNA to tissues, through the systematic periodic introduction of younger primordial whole (nucleated) stem cells and bone marrow into a recipient, resulting in re-establishment of an earlier relative biological clock set-point, with respect to the number of cell generations-divisions are disclosed.

### RELATED APPLICATION

The present application claims priority of U.S. Provisional Application No. 60/923,145 filed Apr. 11, 2007, which is incorporated herein in its entirety by this reference.

### FIELD OF THE INVENTION

The present invention relates to anti-aging compositions.

### BACKGROUND

Autologous stem cells have been found in many adult tissues such as brain, heart, and pancreas, in addition to bone marrow. The major limitation to the therapeutic use of these cells is their relatively quantities and their reduced potential for proliferation. To immortalize such cells, the use of cell feeders and/or growth factor genes to expand stem cell availability raises potential problems with contamination of cell feeders and tumorigenicity. Thus, there is an inherent limitation absent a means for proliferation.

To date, stem cells found in bone marrow, placenta and umbilical cord blood have been used extensively to repopulate the hematopoietic system. The use of cord blood has several unique advantages, including no risk to the donor, low risk of graft-versus-host disease, and rapid availability. However, the major disadvantage of placenta and cord blood transplantation, is the low number of hematopoietic progenitor cells (CD34.sup.+cells) compared with bone marrow or mobilized peripheral blood. Unfortunately, the populations of pluripotent SCs are also limited.

A major limitation to stem cell-based therapy is the need to generate sufficient numbers of cells retaining their pluripotentiality. Cell number can be increased by introduction of growth stimulatory genes to produce sustained expansion. However, loss of differentiation potential and safety concerns has limited the usefulness of this approach. In addition, promoting cell growth in an undifferentiated state by co-incubation with feeder layers increases the risk for cross-transfer of pathogens. Therefore, the use purified recombinant growth factors has been widely applied to expand stem cell cultures. Thrombopoietin (TPO) has been shown to have multiple functions not only inducing differentiation to a platelet and megakaryocytic phenotype, but also stimulating the proliferation of hematopoietic cells (See B. Fishley, W. S. Alexander, Thrombopoietin signalling in physiology and disease, Growth Factors 22 (2004) 151-155; and D. J. Kuter, C. G. Begley, Recombinant human thrombopoietin: basic biology and evaluation of clinical studies, Blood 100 (2002) 3457-3469).

Blood stem cells (derived from bone marrow) may be able to generate both skeletal muscle and neurons. This facility of AS cells to generate specialized cell types of another type of tissue has been variously referred to as “plasticity,” “unorthodox differentiation,” or “transdifferentiation.” Presently, there is evidence that AS cells can generate mature, fully functional cells, or that the cells have restored lost function in vivo. Collectively, studies on plasticity suggest that stem cell populations in adult mammals are not fixed entities, and that after exposure to a new environment, they may be able to populate other tissues and possibly differentiate into other cell types. However, as in other cases there are fundamental limitations on total SC potency.

Fortunately, several new methods have been devised for collecting, screening and proliferating undifferentiated pluripotent SCs from these and other embryonic, placental and umbilical cord sources, and others have been announced. Further, even other major break-throughs in pluripotent collection and proliferation procedures are on the horizon.

Unfortunately, one the greatest therapeutic opportunities for the use of these primordial SCs, when they do become plentiful, has yet to be discovered—e.g. an ability to effectively use them to retard or reverse aging.

Prior art research focuses primarily on utilizing human DNA that is obtained in stem cells harvested from donors other than the intended recipient (non-autologous, i.e. embryonic or fetal stem cells). Still other prior art explores autologous DNA obtained from stem cells collected from the donor in real-time or relatively current to the recipient's age and treatment period. Other art discloses both types involve “injecting” whole stem cells (whether autologous or not) into target tissue to promote repair, such as in spinal cord injury or damaged heart. United States Patent Application, 20040151706, Shakhov, Alex; et al., Aug. 5, 2004, for example provides a Method for treating a cytopathological disease or other medical condition in a mammal, including the steps of harvesting a biological specimen containing stem cells selected from peripheral blood and/or bone marrow from the body of a donor, then storing the harvested bone marrow for a predetermined waiting period before re-infusing it back into the same donor after the donor later contracts and is diagnosed with one or more of a cytopthalogical illness, a chronic fatigue syndrome, and/or damaged issue.

United States Patent Application 20040258673, Hirose, Thomas Gordon; et al., Dec. 23, 2004, “Elective collection and banking of autologous peripheral blood stem cells (Hirose),” is invention using an individual's own peripheral blood stem cells for future healthcare uses. In Hirose an individual can elect to have his or her own stem cells collected, processed and preserved, while he or she is in healthy or “pre-disease” state, for future distribution for his or her healthcare needs. The Hirose process includes collection, processing, preservation and distribution of adult (including pediatric) peripheral blood stem cells during non-diseased state. Hirose stem cells collected will contain adequate dosage amounts, for one or more transplantations immediately when needed by the individual for future healthcare treatments. The Hirose collected adult or non-neonate child peripheral blood stem cells can be aliquoted into defined dosage fractions before cryopreservation so that cells can be withdrawn from storage without the necessity of thawing all of the collected cells. Hirose mentions in his claims at least 2 different collections at 2 different ages/weights U.S. Patent Publication 20070053888, Hariri; Robert J., Mar. 8, 2007, “Use of umbilical cord blood to treat individuals having a disease, disorder or condition (Hariri),” provides methods of using cord blood and cord blood-derived stem cells in high doses to treat various conditions, diseases and disorders. Hariri's high-dose cord blood and cord blood-derived stem cells have a multitude of uses and applications, including but not limited to, therapeutic uses for transplantation and treatment and prevention of disease, and diagnostic and research uses. In particular, Hariri's cord blood or cord blood-derived stem cells are delivered in high doses, e.g., at least 3 billion nucleated cells per treatment, where treatment may comprise a single or multiple infusions. The Hariri provides for the use of cord blood or cord blood-derived stem cells from multiple donors without the need for HLA typing.

U.S. Patent Publication 20040197310, Paul R.; et al. Oct. 7, 2004, “Compositions and methods for using umbilical cord progenitor cells in the treatment of myocardial infarction (Sanberg),” provides compositions and methods for treating circulatory disorders, for treating myocardial infarctions, for producing cardiac muscle cells, and for treating injured tissue in an individual. More particularly, the present invention provides methods of treating circulatory disorders by administering an effective amount of a composition comprising an umbilical cord blood cell. In one Sanberg embodiment, the circulatory disorder is myocardial infarction.

U.S. Patent Publication 20060275271, Chow; Robert, Dec. 7, 2006, “PLASMA-DEPLETED, NON-RED BLOOD CELL-DEPLETED CORD BLOOD COMPOSITIONS AND METHODS OF USE (Chow),” provides umbilical cord blood (UCB) compositions that possess the unique features of having plasma that is substantially depleted from the UCB unit and red blood cells (RBC) that are not depleted from the UCB unit. Such UCB units can be prepared by a process that combines plasma depletion with cryopreservation, selection, thawing, and/or transplantation of hematopoietic stem cells to provide superior clinical outcome by maximizing post-processing cell recovery and post-thaw infusion cell dose. Methods for treating a wide variety of malignant diseases and benign diseases associated with the hematopoietic system by administering the UCB compositions of the present invention are also provided.

The prior art (teaching stem cell, cord blood, bone marrow replacement) generally teaches prevention of disease and therapies and compositions are reactive—generally providing treatment after the onset of a disease. The prior art does do not teach proactive periodic primordial (preferably pluripotent) stem cell use with or with bone marrow infusion, much less at a frequency with dosages and conditions capable of affecting a global cellular renewal of all or essentially all cell tissue. The Hariri reference teaches progenitor cells, without suggesting a combination of stem cells with bone marrow in an ex-vivo composition, or a systematic/periodic infusion protocol.

Applicant can find no reference of an anti-aging method or composition employing proliferated primordial stem cells, preferably pluripotent. Nor does the Hirose autologous reference (representing an alternate healthcare insurance system) provide for periodic proactive infusion.

While the prior act incidentally recognizes ‘renovating’ or rejuvenating the tissues from stem cells, it does not distinguish on the requisite usage of biologically younger primordial stem cells for age reduction. Rather, the art principally deals with stem cells targeted at diseases and specific organs (the brain, the heart, the muscles, localities, end tissues, etc.).

### SUMMARY OF INVENTION

The invention relates to an integration of 1) anti-aging compositions incorporating bone marrow, stem cells and combination, with methods of collecting, proliferating and manufacturing same, together with 2) the periodic systematic infusion of these compositions into a recipient, where recipient experiences the establishment of an earlier relative biological clock set-point within his body tissue, with respect to the number of cell generations-divisions. This is seen in the formation of a transient tissue chimera containing two distinct DNA chronological &/or biological aged tissues, where the younger tissue eventually out-replicates, transmogrifying the older tissue, maintained through the continuation/systematic series of systemic introductions (infusion) containing primordial stem cells (SC) and optionally bone marrow (BM) or both.

More specifically, the invention resides in the use of primordial stem cells (preferably pluripotent stem cells) and optionally (or both) bone marrow together with a proactive systematic systemic means of introductions (infusion), wherein a global inchoate-transient tissue chimera is formed ever evolving toward a mature tissue chimera, characterized by markedly younger cellular tissues biologically relative to the initial chronological and biological age of the recipient. In other words, Applicants' invention provides for a global renewal of cellular structures with biologically younger tissue throughout recipient's entire body, whereby the natural chronological aging process of recipient is retarded or reversed.

Donor primordial totipotent, pluripotent and omnipotent stem cells, which are matched, do not require HLA typing, transmogrified and otherwise (non graft-versus-host rejecting-NGvHR) will be employed in order to avoid autoimmune response. The art suggests that primordial SCs are largely absent host immune responses and thus should be largely void of this concern. However, in certain cases in the practice of this invention immune suppression is expressly contemplated. See, e.g., U.S. Publication Nos. 20050123525, 20060159666,

This invention incorporates both autologous and allogeneic methods, wherein the delivery of stem cells of each practice includes markedly younger DNA than the chronological age of recipient and a means to cause the formation of a transient tissue chimera.

An important element of the invention is an ex-vivo composition of compatible (non graft-versus-host rejecting—NGvHR) primordial SCs and bone marrow (BM), activated to enhance the formation of the transient tissue chimera.

Collection of stem cells from a living being (donor) is provided, preferably i) after donor's conception (absent damage to embryo), ii) umbilical cord and/or placenta collection, iii) and/or collection at birth through early life of a donor, are all contemplated. The collection of bone marrow is also an essential element. The preferred stem cell (and bone marrow) collection includes collecting as much as possible before donor's chronological age of 10. Autologous practice additionally includes stem cell and donor collection through out donor-recipient's life.

Augmentation and/or proliferation are important elements and are employed to expand stem cell and where possible bone marrow populations. Compositions and procedure for infusing same and compatible compositions thereof designed to avoid “graft versus host rejection” are essential in this practice and disclosed in the detailed description below.

Young primordial stem cell collection, preferably post conception and at birth, and the proliferation of these SC represent an especially essential element of this invention. Post conception collection of SC are made under the proviso that no damage is ever done to an embryo.

An essential element of the invention is a routine/systematic and periodic transportation (infusion) program for delivering of these anti-aging compositions into recipient such that a transient tissue chimera is formed and maintained. Infusions are contemplated throughout recipient's entire life, probably not beginning much earlier than age 13 years of age.

This life long infusion regime will become a routine proactive regime consistent with recipient's age, weight, gender, and results desired. Infusion may be either orally, vascular and/or directly into recipient's bone marrow, or a combination. Infusion therapy contemplates single point entry or multiple point entry. Enhancement methods may also be employed, such as signally, stimulation, targeting and the like to increase bone marrow production of SC's after infusion and/or to target certain tissues.

The allogeneic practice of this invention resides in the collection of stem cells of a biologically younger donor, preferably embryonic SCs, at birth (cord or placental SC), and/or before age 10 SCs. These cells will be accumulated, separated, augmented and proliferated to achieve desired quantities and compositions necessary to achieve the objective of applicants' invention.

Stem cell proliferation, particularly contemplates growth of the preferred totipotent and pluripotent stem cells. In the autologous and allogeneic practices young stem cells and bone marrow samples will be stored and preserved, as required.

Thus, the essence of the invention resides in the collection of young DNA containing primordial stem cells (and bone marrow), the proliferation of said stem cells to provided the necessary quantities needed for a period infusion program, and the proactive periodic transfusions of these compatible stem cells, preferably together with bone marrow whereby said infusion program results in an adsorption of younger DNA containing stem cells throughout recipient's entire body, forming a transient tissue chimera—regenerating all cellular, tissue, bone, nerve, muscular and organic structure of recipient with said younger infused DNA.

The practice of this invention contemplates a wide range of mammals, preferably humans.

### DETAILED DESCRIPTION OF INVENTION

Applicant's invention is predicated on 5 fundamental elements/steps:
1. Periodic collection of stem cell and/or bone material from a donor, whereby the mass of all collected samples has an average biological age ranging from after conception to 70 years +/− of age, with preferred biological age ranging from after conception to under 50, and a more preferred range from after conception to birth (umbilical cord) to under chronological age 10. Collection/harvesting optionally commence as soon as possible after conception as possible (absent damage to the embryo).
2. Concentration/sorting, augmentation and proliferation of stem cell (and/or bone marrow) such that resultant product is compatible with recipient (as provided below) and of sufficient quantities to be used routinely on a regular/periodic infusion schedule. Preservation and storage as needed. When providing for extended period of storage, storage must secure, appropriately logging, cataloging and controlling as required, does not cause or result in any appreciable aging or disability to samples, and that after storage said stem cell samples and/or bone marrow may be proliferated and augmented as necessary. Augmentation and/or proliferation are expressly contemplated after some period of storage, if any, prior to infusion.
3. Creation of infusion compositions comprising SCs and/or bone marrow.
4. Periodically on a proactive routine transfusion, infusion or grafting (infusion) said SC/BM compositions into the donor after recipient achieving a minimum age normally not less than 13 years of age, which can benefit from said infusion, anticipated to be periodically over recipient's entire lifetime. The infusion compositions contemplates minimum concentrations of primordial totipotent or pluripotent stem cells (and optionally omnipotent) for minimum potency. Transformation of said SC's in the manufacture of Applicant's BM compositions is anticipated, wherein certain differentiated SC product many be generated. Such primordial SC differentiation may be inhibited or enhanced.
5. The proactive periodic infusion regime will result in biologically younger transfused stem cells causing a global cellular integration of a body wide transient tissue chimera, which is characterized by the collective tissue composition of chronologically older DNA-containing cells being renewed and transmogrified by the biologically younger DNA-containing infused stem cells.

After each infusion or series of infusions expected biologically age differentials of the resulting cellular transient tissue chimera structure will contain younger tissue at least 3 months, 6 months, 1 year, 2, 3, 4, 5 to 10 years younger (or greater) than the original biological/chronological age of the recipient's cellular structure/tissue (prior to infusion) or the chronological age of recipient, comprising the balance of the chimera—until the tissue is substantially or essentially totally replaced.

The tissue chimera will range from an inchoate to a mature tissue with two or more age different tissues. Each series of younger DNA infusion regimes (assuming each regime contains the same age DNA) will non-the-less result in variable ages within any given chimera. After a prolonged continuation of infusions of sufficient duration (and dosage) an inchoate-transient tissue chimera will advance into a more mature chimera, where the younger cells eventually transmogrify the older cells becoming the majority or preponderance of the chimera. Different tissues groups (e.g. skin, heart, nerves, bone, brain, etc) will experience difference rates of turn over under different time lines. Thus, it is expected that different tissue chimera (e.g. different organs) will achieve maturity (wherein a majority or preponderance of tissue is younger compared to the older/oldest tissue; or alternatively where a blend of younger tissue replaces the older tissue, and wherein this blend of younger tissue become a majority of the chimera). Naturally, with this being a life-long program, the chimeras will experience different combinations of younger tissue transmogrifying prior infused young chimera tissue (formerly representing the younger tissue, but now becoming the middle aged tissue, and eventually becoming the older aged tissue).

Naturally, there are many derivatives and variations of this process, which embody the aforementioned elements, and included in the appended claims.

As provided herein infusion and transfusion include injection, transplantation, grafting and/or implantation (infusion), and any other means of delivering SCs and/BM into recipient. This includes a periodic initial regime and active routine of cellular/tissue maintenance after an initial regime. This routine of infusions is systematic preventative care and administered proactively prior to the onset of any disease, which may be contracted by recipient. It is one of a global regeneration of the body's entire cellular tissue to a early biological clock set point.

Basically, this invention embodies a elegant and unique delivery system to replace the body's older cell structures throughout the entire body with younger more vital DNA containing cells, resulting in a universal transformation of tissue structures and organs (on a global basis) into a younger version.

Mammalian cells are pre-programmed, through their genome (DNA mechanism) so as to undergo a finite number of cellular replications—divisions. As the individual cells (which define the total tissue structure) progress along their continuum of number of cellular divisions, their rate of replication slows. Eventually, signs of aging become apparent, as damaged and worn out tissue cells are not timely replaced. Cells become worn, exhausted, damaged from environmental insults, and deprived of necessary co-factors and interactive influences from other failing bodily endocrine and physiologic functions.

Stem cells are cells with the ability to divide for indefinite periods in culture and to give rise to specialized cells. Stem cells are a self-renewing population of cells that can be found predominately within the bone marrow, but can be found in blood in other parts of the body (peripheral stem cells) in certain circumstances. Stems cells can also be found in a newborn's placenta and cord blood at time of delivery.

While research efforts are and have been underway to intervene in the aging process, none propose to simply replace the entire body's cells with younger, fresh cells through a self-adaptive (autologous) grafting process, employing the recipient's very body and DNA—in essence a younger self-cloning process of integrating the old until the new can take over.

Human stems cells contain the body's complete genetic information, factors, determinants and capacity for generation of differentiated tissues throughout the entire body of a person into specific end-organ and support tissues such as bone, nerves, muscle, heart, liver, brain, endocrine organs, skin etc.

Mammalian stems cell can be collected, stored and re-infused at a later date into the bloodstream of the same being from which they were harvested at an earlier time in his/her life. They will disseminate throughout the entire body, lodging in all tissues. Interacting with factors and determinants specific to differentiated ‘resident’ tissue cells (already evolved to a specific and distinguishable type of tissue such as bone or muscle, etc.), the stem cells will be induced through interactions with the adopted tissues to ‘differentiate’ or evolved into the respective distinguishable tissues in which they find residence forming a graft and transient chimera of older and younger DNA-containing cells.

Possessing younger DNA, vital enzymes and co-factors, these younger stem cells will divide at faster rates than their adopted perspective resident mature host tissue cells and then mature into new and younger host cells, “replacing” the older resident cells, eventually resulting in an essentially homogeneous cellular composition with respect to the age of the cells' DNA (thus establishing a younger total body).

Infusion with sufficient quantities of vital stem cells over regular, adequate and periodic time-frames would result in successive, but eventually diminishing replacement of aging tissue. Because, it is not known what tissues may have an inherent resistance to ‘replacement’; it is not known what quantities of stem cells would actually be needed and what time period it would take for total tissue replacement with the younger newly differentiated cells; and while it might be presumed based upon natural statistical truths, it is not known if there is a dose-time response curve (more stem cells infused—the faster they take over by replacing the older tissue cells). In addition, one's supply of viable stem cells would eventually be exhausted, as would their and perhaps society's will and resources. An endless supply of one's younger stem cells could not be obtained to be indefinitely accessed by a person later in his/her life.

### Collection of Primordial Stem Cells (SCs)

Collection and use of proliferated primordial SCs, particularly totipotent, pluripotent and omnipotent SCs is an express requirement/embodiment of this invention. Pluripotent SCs are the preferred SCs of this invention. Numerous methods are contemplated and are provided in the art. For example, United States Patent Application 20040120932, Zahner, Joseph Edward, Jun. 24, 2004, discloses methods for deriving adult pluripotent stem cells from fully differentiated adult somatic cells by in vitro nuclear remodeling; US Patent publication 20020188963 provides for an isolated population of embryonic stem (ES) cells and methods of obtaining these ES cells. In one aspect, the target ES cells are obtained by co-culturing embryo cells from a target with non-target ES cells.

Applicants' invention includes Stem cell collection employing various means directly from an embryo, placenta, cord, cord blood, donor's bone marrow, donor's vascular blood stream, donor vascular department, donor tissue and organs, donor feces, from fetal, neonate and other sources. Donor's may be 3^{rd} party donors or autologous (donor-recipient).

Recent breakthroughs permit collection of ES cells directly from live embryos absent damaging fetuses. Other means exist including that disclosed in US Patent Publication 20060084168 20030213008 Still others are disclosed below.

Placental stem cell collection provides an excellent resource for totipotent and pluripotent stem cell collection. It has been report that the placenta used as a bioreactor for endogenous cells, can provide various pluripotent and/or totipotent stem cells, by incubating the placenta for 48 hours with perfusate solution. See U.S. Pat. No. 7,045,148, US Patent Publication 20030032179

The conventional placenta technique for SC collection is similar to that of cord blood. It is based on the use of a needle or cannula which is used with the aid of gravity to drain the cord blood from the placenta. Usually the needle or cannula is placed in the umbilical vein and the placenta is gently massaged to aid in draining the cord blood from the placenta. Thereafter the drained placenta has been considered to be of no use and has typically been discarded. A major limitation of stem cell procurement from cord blood has been the frequently inadequate volume of cord blood obtained resulting in insufficient cell numbers to reconstitute bone marrow after transplantation. The method requires access to freshly drained human placentas which have been subjected to a conventional cord blood recovery process by draining substantially all of the cord blood from the placenta. It is important that the placenta be properly stored and drained if it is to be a suitable source of embryonic stem cells. Generally, a placenta should be stored in an anticoagulant solution at a temperature of 5 to 25.degree. C. (centigrade) for no more than 48 hours prior to the collection of the cord blood. Suitable anticoagulant solutions are well known. An acceptable anticoagulant solution comprises a solution of heparin (1% w/w in 1:1000 solution). Generally, the drained placenta should be stored for no more than 36 hours before the embryonic-like stem cells are collected.

Acceptable embryonic-like stem cells (acceptable to Applicants' invention) may be extracted from placenta employing a method is based on the perfusion of the drained placenta with a suitable aqueous fluid such as an anticoagulant dissolved in any suitable aqueous isotonic fluid such as 0.9N sodium chloride solution. The anticoagulant for the perfusion liquid may comprise heparin or warfarin sodium at a concentration which is sufficient to prevent the formation of clots of any residual cord blood. Generally from 100 to 1000 units of heparin may be employed.

The extraction procedure is based on the passage of the perfusion liquid through either or both of the umbilical artery and umbilical vein using a gravity flow into the placenta which is suspended in such a manner that the umbilical artery and umbilical vein are at the highest point. It is preferred to connect the umbilical artery and the umbilical vein simultaneously to a pipette that is connected via a flexible connector to a reservoir of the perfusion liquid which is passed into the umbilical vein and artery and collected in a suitable open vessel from the surface of the placenta that was attached to the uterus of the mother during gestation.

The collection technique is based on the use of a sufficient amount of the perfusion liquid that will result in the collection of the cells left after the drainage of the cord blood. It has been observed that when the perfusion liquid is first collected, the liquid is colored with the residual red blood cells and tends to become clear as the perfusion liquid is passed through the placenta. Generally from 30 to 100 ml of perfusion liquid is adequate to collect the embryonic-like cells but more or less may be used depending on the observed results.

In one embodiment, the placenta may be used as a bioreactor for endogenous cells, including but not limited to lymphocytes and various kinds of pluripotent and/or totipotent stem cells, by incubating the placenta for 48 hours with perfusate solution.

Other methods of collecting placenta SCs are contemplated in Applicants invention, including those provided in U.S. Pat. No. 7,045,148, Hariri (May 16, 2006) and others are provided in US Patent Publications 20070043328, 20050272148 20020123141, 20050276792, 20050148034, 20050118715, 20040161419, 20040028660 20030235909 20020160510

In the practice of Applicants' invention human stem cell compositions derived from bone marrow and blood are contemplated. Initially, bone marrow cells may be obtained from a source of bone marrow, e.g., iliac crests, tibiae, femora, spine, or other bone cavities. Other sources of human hematopoietic stem cells include embryonic yolk sac, fetal liver, fetal and adult spleen, blood, including adult peripheral blood, placenta and umbilical cord blood.

For isolation of bone marrow from fetal bone or other bone source, an appropriate solution may be used to flush the bone, which solution will be a balanced salt solution, conveniently supplemented with fetal calf serum or other naturally occurring factors, in conjunction with an acceptable buffer at low concentration, generally from about 5-25 mM. Convenient buffers include Hepes, phosphate buffers, lactate buffers, etc. Otherwise bone marrow may be aspirated from the bone in accordance with conventional ways.

Bone marrow contains three stem cell populations, hematopoietic stem cells, bone marrow stromal cells, and (possibly) endothelial progenitor cells. Bone marrow stromal cells are a mixed cell population of cells that generate bone, cartilage, fat, fibrous connective tissue, and the reticular network that supports blood cell formation (mesenchymal stem cells of the bone marrow also give rise to these tissues, and may constitute the same population of cells as the bone marrow stromal cells). Studies of hematopoietic stem cells from bone marrow demonstrate an ability to regenerate an entire tissue system, i.e., all cell types found in blood. Thus, bone marrow shows promise as a source for AS cells exhibiting plasticity, and further development of materials and techniques may allow the utilization of all three stem cell populations found in bone marrow.

Bone marrow compositions substantially free of cells dedicated to a particular lineage, cells carrying markers associated with lineage dedication, wherein the stem cells are able to regenerate and differentiate to populate the various hematopoietic and/or non-hematopietic lineages, are acceptable. A substantially homogenous composition may be obtained by selective isolation of cells free of markers associated with differentiated cells, while displaying epitopic characteristics associated with the stem cells, and by regeneration of the isolated stem cells in defined culture systems leading to different hematopoietic and/or non-hematopietic cell lineages.

The stem cells are characterized by both the presence of markers associated with specific epitopic sites identified by antibodies and the absence of certain markers as identified by the lack of binding of certain antibodies. It is not necessary that selection is achieved with a marker specific for stem cells. By using a combination of negative selection (removal of cells) and positive selection (isolation of cells), a substantially homogeneous stem cell composition can be achieved.

A large proportion of differentiated cells to isolate primordial SCs may be removed by initially using a “relatively crude” separation. The source of the cells may be the bone marrow, fetal, neonate or adult or other hematopoietic cell source, e.g., fetal liver, peripheral blood, umbilical cord blood, and the like. For example, magnetic bead separations may be used initially to remove large numbers of lineage committed cells, namely major cell populations of the hematopoietic systems, including such lineages as T-cells, B-cells, (both pre-B and B-cells), myelomonocytic cells, or minor cell populations, such as megakaryocytes, mast cells, eosinophils and basophils. Desirably, at least about 70%, usually at least 80% of the total hematopoietic cells will be removed. It is not essential to remove every dedicated cell class, particularly the minor population members at the initial stage. Usually, the platelets and erythrocytes will be removed prior to sorting. Since there will be positive selection in the protocol, the dedicated cells lacking the positively selected marker will be left behind. However, it is preferable that there be negative selection for all of the dedicated cell lineages, so that in the final positive selection, the number of dedicated cells present is minimized. The stem cells are characterized by being for the most part CD34.sup.+, CD3.sup.−, CD7.sup.−, CD8.sup.−, CD10.sup.−, CD14.sup.−, CD15.sup.−, CD19.sup.−, CD20.sup.−, CD33.sup.−, and Thy-1.sup.+. A highly stem cell concentrated cell composition is CD34.sup.+, CD10.sup.−, CD19.sup.− and CD33.sup.−, more particularly in addition CD3.sup.− and CD8.sup.−, preferably in addition Thy-1.sup.+. The CD3.sup.−, 8.sup.−, 10.sup.−, 19.sup.−, 20.sup.− and 33.sup.−. The CD10/19/20 markers are associated with B-cells, CD3/4/8 markers are associated with T-cells, CD14/15/33 cell markers are associated with myeloid cells. The Thy-1 marker is absent on human T-cells. Also, for human CD34.sup.+, rhodamine 123 can divide the cells into high and low subsets. See Spangrude, (1990) Proc. Natl. Acad. Sci. 87, 7433 for a description of the use of rhodamine 123 with mouse stem cells. Preferably these cells are rhodamine low. However, is non-the-less necessary to isolate the rare pluripotent human stem cell from the other cells contained in bone marrow and other hematopoietic sources.

Morphologic evaluation of the 34+Thy+Lin− cells indicates that the multipotent progenitors, “stem cells” are of medium size. Light scatter evaluation shows that “stem cells” have a blast cell profile with low side scatter. These observations indicate that the “stem cells” have a unique density profile. It has been found that the low density fractions from density fractionated human bone marrow are enriched for CD34+Thy+Lin− cells.

Various techniques may be employed to separate the cells by initially removing cells of dedicated lineage. Monoclonal antibodies are particularly useful for identifying markers (surface membrane proteins) associated with particular cell lineages and/or stages of differentiation. The antibodies may be attached to a solid support to allow for crude separation. The separation techniques employed should maximize the retention of viability of the fraction to be collected. For “relatively crude” separations, that is, separations where up to 10%, usually not more than about 5%, preferably not more than about 1%, of the total cells present having the marker, may remain with the cell population to be retained, various techniques of different efficacy may be employed. The particular technique employed will depend upon efficiency of separation, cytotoxicity of the methodology, ease and speed of performance, and necessity for sophisticated equipment and/or technical skill.

Procedures for separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., plate, or other convenient technique. Techniques providing accurate separation include fluorescence activated cell sorters, which can have varying degrees of sophistication, e.g., a plurality of color channels, low angle and obtuse light scattering detecting channels, impedance channels, etc.

One procedure which may be used is in a first stage after incubating the cells from the bone marrow for a short period of time at reduced temperatures, generally about 4.degree. C., with saturating levels of antibodies specific for a particular cell type, e.g., CD3 and 8 for T-cell determinants, the cells may then be washed with a fetal calf serum (FCS) cushion. The cells may then be suspended in a buffer medium as described above and separated by means of the antibodies for the particular determinants, using various proteins specific for the antibodies or antibody-antigen complex.

Conveniently, the antibodies may be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular cell type. Any technique may be employed which is not unduly detrimental to the viability of the remaining cells.

Other procedures are found in the art for collecting the SCs of Applicants' invention from bone marrow sources. See generally for example, U.S. Pat. Nos. 5,914,108, 5,763,197, 5,750,397, 5,739,110, 5,716,827, 5,677,136, 5,658,761, 5,087,570, 5,061,620, 5,460,964, 5,436,151, 5,523,286, 5,643,741; US Patent publications 20060018889 20050118144 20050164380 20030017588.

Cord and Cord Blood Stem Cell Collection is also contemplated and known in the art. For example see US Patent Publication 20060205071, 20040136967. There are a number of methods known in the art for the collection of SCs, which may be used in the practice of Applicants invention.

Generally the STEM CELLS implicated in Applicants' invention include (but are not limited to) embryonic stem cells, embryonic totipotent stem cell, embryonic pluripotent stem cells, embryonic omnipotent (multipotent) stem cells, embryonic progenitor stem cells, bone-derived stem cells, bone marrow/cord derived pluripotent stem cells, bone marrow/cord derived omnipotent stem cells, bone marrow/cord derived progenitors, hemeangioblast stem cells, angiohematopoietic stem cells, multipotent adult progenitor cells, tissue-derived pluripotent stem cell, muscle-derived stem cells, fat-derived stem cells, mesenchymal stem cells, neural stem cells, multipotent adult progenitor cells (MAPCs), vascular pericytes, blood or marrow derived CFU-GEMM, blood or marrow derived CD34.sup.+stem cells or hematopoietic stem cells, CFU-blast, satellite cells (skeletal myoblast progenitors), angioblasts (endothelial cell progenitors), mesenchymal stem cells, embryonic stem cells. Some of these stem cells, e.g., neural stem cells, CD34+ cells or multipotent adult progenitor cells can differentiate into one or more of osteoblasts, chrondroblasts or chondrocytes, fibroblasts; adipocytes, skeletal myoblasts, smooth muscle cells, cardiac myocytes, endothelial cells, hepatocytes, neurons, glial cells, astrocytes or oligodendrocytes. The preferred practice however includes primordial stem cells, selected from totipotent, pluripotent and multipotent. The most preferred by totipotent and pluripotent.

Bone marrow harvesting/collection is also an embodiment of Applicants' invention. Traditionally, bone marrow has been harvested using a bone marrow puncture needle, such as Thomas needle, to puncture ilium. The bone marrow puncture needle is constructed in such a manner as to insert and fit an inner needle into a tubular mantle while allowing the tip of the inner needle to project from the mantle. The bone marrow puncture needle, which is equipped with a handle, percutaneously punctures ilium, and once the tip of the needle reaches bone marrow, the inner needle alone is drawn out and the mantle is kept indwelling the bone marrow to be subsequently used. As a method for harvesting bone marrow using this bone marrow puncture needle, typically, an aspiration method is employed. The aspiration method is such that the mantle of the bone marrow puncture needle is connected to a syringe to collect bone marrow by virtue of the aspiration force. Other methods include a bone marrow perfusion method such that two bone marrow harvesting needles are kept indwelling respective ends of a long bone, such as humerus, with one of the needles connected to an injection syringe and the other connected to a centrifugal tube for harvesting bone marrow via a collection tube, so as to perfuse bone marrow with a medium such as sterilized and heparinized phosphate-buffered physical saline, and thereby a required quantity of bone marrow is harvested into a collection container at a time by injecting the medium slowly into the bone marrow in such a manner as to wash away the bone marrow. This method is desirable for long bone marrow harvesting, because the needle equipped with a drill is capable of providing a more powerful rotational power than a handle. Methods of harvesting and storing BM are provided in U.S. Pat. Nos. 7,008,394 and 5,456,267.

ENGINEERED BM is also contemplated within the practice of Applicants' invention. In certain instances the use of engineered bone marrow may be preferred. See for example: United States Patent Application, 20060134224, Flake; Alan W.; et al., Jun. 22, 2006, which provides for engineered bone marrow compositions comprising of bone marrow cells, pulverized bone, and type 1 collagen, which can be transplanted into the portal system of a patient. The engineered bone marrow provides a microenvironment, for engraftment of hematopoietic stem cells. It is anticipated that engineered BM may become a meaningful source of compatible BM for the practice of Applicants invention. Certainly, substitutes and artificial ingredients that accomplish Applicants object are contemplated. Engineered BM may have significant advantages in avoiding “grant v host rejections.”

SEPARATION will be required of SCs at some point after their collection (optionally after their storage). Collected stem cells will often be contained with other cells and other SCs, which are also collected. Separation must occur and fortunately separation technique also includes opportunities to improve both the quality of the SC material used in the invention, but also eases subsequent proliferation, manufacturing, handling costs and subsequent infusion costs. Separation and other technique anticipated are know in the art. See for example US Patent Application 20030199050 providing for cell separation using electric fields.

PLURIPOTENT STEM CELLS are stem cells that may be obtained from sources such as embryonic tissues, placenta, cord blood, bone marrow, and certain other sources. U.S. Pat. No. 6,200,806 describes pluripotent stem cells obtained from human embryonic tissue. These cells are preferred in the practice of Applicants invention because they are capable of proliferating in vitro without significant karyotype changes while maintaining a capacity to differentiate into endoderm, mesoderm, and ectoderm tissues. These cells are negative for the SSEA-1 marker, positive for the SSEA-4 marker, and express alkaline phosphatase activity. These cells have euploid karyotypes and none of the chromosomes are obviously altered. U.S. Pat. No. 5,843,780 describes a purified preparation of primate embryonic stem cells that is capable of proliferation in an in vitro culture for and maintains a karyotype in which all the chromosomes characteristic of the primate species are present and not noticeably altered through prolonged culture. These cells maintain a potential to differentiate into derivatives of endoderm, mesoderm, and ectoderm tissues throughout the culture. These cells will typically not differentiate when cultured on a fibroblast feeder layer and they can differentiate to trophoblasts and produce chorionic gonadotropin when cultured at a high density.

Pluripotent cells have been obtained from preimplantation embryos of several animals, e.g., Evans, et al., Theriogenology 33(1):125-128, 1990; Evans, et al., Theriogenology 33(1):125-128, 1990; Notarianni, et al., J. Reprod. Fertil. 41(Suppl.):51-56, 1990; Giles, et al., Mol. Reprod. Dev. 36:130-138, 1993; Graves, et al., Mol. Reprod. Dev. 36:424-433, 1993; Sukoyan, et al., Mol. Reprod. Dev. 33:418-431, 1992; Sukoyan, et al., Mol. Reprod. Dev. 36:148-158, 1993; lannaccone, et al., Dev. Biol. 163:288-292, 1994).

Human embryonic carcinoma cells, which are pluripotent cells obtained from teratocarcinomas resemble human embryonic stem cells (Andrews, et al., Lab. Invest. 50(2):147-162, 1984; Andrews, et al., in: Robertson E., ed. Teratocarcinomas and Embryonic Stem Cells: A Practical Approach, Oxford: IRL press, pages 207-246, 1987). Embryonic carcinoma cells can be induced to differentiate in culture, which is characterized by the loss of specific cell surface markers (SSEA-3, SSEA-4, TRA-1-60, and TRA-1-81) and the appearance of new markers. As provided above Pluripotent SCs can be found in BM, placenta, cord, cord blood, certain tissue and other sources. See 20060216821. Applicants' invention expressly contemplates protocols and technique for the isolation and collection of pluripotent SCs.

An optimal Pluripotent SC source includes EMBRYONIC stem (ES) cells, which can proliferate indefinitely in an undifferentiated state. Furthermore, ES cells are readily easy to separate into primordial totipotent or pluripotent cells. These primordial SCs can generate into all of the cells present in the body (bone, muscle, brain cells, etc.). ES cells have been isolated from the inner cell mass of the developing murine blastocyst (Evans et al., Nature 292:154-156, 1981; Martin et al., Proc. Natl. Acad. Sci. U.S.A. 78:7634-7636, 1981; Robertson et al., Nature 323:445-448, 1986; Doetschman et al., Nature 330:576-578, 1987; and Thomas et al., Cell 51:503-512, 1987; U.S. Pat. No. 5,670,372). Additionally, human cells with ES properties have recently been isolated from the inner blastocyst cell mass (Thomson et al., Science 282:1145-1147, 1998) and developing germ cells (Shamblott et al., Proc. Natl. Acad. Sci. U.S.A. 95:13726-13731, 1998) (see also U.S. Pat. No. 6,090,622, WO 00/70021 and WO 00/27995).

REVERSE GENETIC TRANSITION/ENGINEERING of committed (omnipotent or progenitor) SCs back into pluripotent SCs form is also contemplated [cites ______] PROLIFERATION OF PRIMORDIAL SC is an essential embodiment.

Applicant's invention is predicated upon one or more methods of proliferating collected primordial SCs, especially pluripotent SCs (while all SCs may be included). Existing proliferation methods are now available, which appear able to achieve Applicants' quantity needs to practice this invention, and more processes are expected. See for example, United States Patent Application 20070010011, Parsons; Xuejun Huang; et al., Jan. 11, 2007, which provides for cultivation in serum-free, feeder-free, and conditioned-medium-free—the long-term growth of undifferentiated pluripotent stems. Also, see 20060030040, Yang; Mei-Ju; et al. Feb. 9, 2006, which discloses a method of culturing embryonic stem cells that remain substantially undifferentiated while maintaining their pluripotency to differentiate into subsequent germ layer cells. Others provide for pluripotent SC proliferation from bone marrow culture, see 20070077201, 20060134784. Still others from placenta, cord and cord blood, see 20070059824, 20060134784. 20060078993. Also, see US Patent Applications 20060216821, 20060127370, 20050221480, 20040087016, 20030212024, 20030186439. In 20060127370, Niwa; Hitoshi; et al., Jun. 15, 2006, provides for the proliferation of undifferentiated pluripotent stem cells that retain their differentiation potency by culturing the pluripotent stem cells in a medium free of a feeder cell, or a serum. This is attained by using a culture medium for pluripotent stem cells, which is supplemented with an inhibitor of an adenylate cyclase activity. In United States Patent Application, 20060030041, Furcht; Leo T., et al., Feb. 9, 2006, provides for isolated stem cells of non-embryonic origin in their undifferentiated or differentiated state to form cells of multiple tissue types.

Thus, proliferation of placenta, cord, cord blood, bone marrow and vascular department derived pluripotent SCs is expressly contemplated in the invention. See U.S. Pat. No. 5,914,108, Publications 20060134784 and 20070059824, with the later disclosing methods of providing for umbilical cord blood-derived pluripotent fibroblast-like-macrophages.

The AUGMENTATION step—like the proliferation step, may be exercised at any stage of the invention, including after collection, prior to storage, after storage, in the manufacture of the sera contain SCs, or prior to infusion.

Numerous forms of augmentation and augmentation methods are anticipated. Unlimited examples include augmentation to increase SC potency, reduce weaker SC product, increase population of stem cells, reduce or reverse differentiation or differentiation potential, specialized differentiation/tissue genetic targeting, avoiding NGvHR or immune response desensitizing, initiation of primordial SC proliferation, initiation of SC division, signaling, targeting, and the like. An example of a hematopoietic augmenting method includes United States Patent Application 20030054549, Takebe, Minoru; et al., Mar. 20, 2003, which provides a stem cell-augmenting material capable of augmenting hematopoietic stem cells of bone marrow, and embryonic stem cells—using isoflavone aglycone (which has estrogen-like activity) that does not block the enzyme activity of cell proliferation factor. Also see U.S. Patent Publication Nos. 20060233840 20060034767 20030022249 20020183297 20060068414

STORAGE AND STEM CELL BANKING, logging, security and other measures insuring preservation and storage (long term and short term) are contemplated. Numerous art references incorporate the elements of the requisite system(s), which must be employed in this invention. It is known that Stem cells are routinely cryopreserved (see e.g., Boyse et al., U.S. Pat. No. 5,004,681, “Preservation of Fetal and Neonatal Hematopoietic Stem and Progenitor Cells of the Blood”, Boyse et al.,) See U.S. Pat. Nos. 5,192,553 and 20050106554, with the later providing for cryopreservation of pluripotent stem cells. Other art provides for extended SC preservation at ambient temperature. Also see US Patent Publications 20060167401 20050276792, 20040258673 20060233768 20060095319 20060063141 20060060494 20040258673 20030220244 20030054331 20020094550

STEM CELL CULTURING, MAINTENANCE, GROWTH AND PRESERVATION METHODS—The culturing, maintenance, preservation, enhancement, growth and storage of collected BM and SC material, cultures, and combination BM/SC compositions (including Applicant's “Activated” BM/SC composition) are an important element of this invention. The art provides numerous methods of variously achieving of these objectives, which are incorporated in full by reference. Incorporated referenced US patents include U.S. Pat. Nos. 6,673,904, 6,667,391, 5,914,108 and incorporated referenced US Patent Publications (not intended to be limiting) include: 20070078113, 20070073186, 20070072830, 20070072293, 20070059826. 20070054258 20030166894 20020132224 20030180784 20070053890, 20070050218, 20070048726, 20070048350, 20070042341 20070042337 20070026042 20070026036 20070025961 20070015830 20060293724 20060292689 20060280727 20060275886 20060270038 20060269907 20060258003 20060246043 20060223050 20060210544 20060204482 20060200043 20060188867 20060167401 20060148017 20060142818 20060134070 20060134069 20060129225 20060127375 20060122373 20060116603 20060094693 20060093999 20060083720 20060078872 20060063738 20060063719 20060057137 20060040894 20060034941 20060024657 20060024386 20060019234 20070050218 20060292689 20060167401 20060122373 20040254560 20040241167 20050233301 20050232965 20050232964 20050229264 20050222191 20050220836 20050214377 20050209688 20050208092 20050202059 20050187608 20050182485 20050181997 20050170019 20050158360 20050147692 20050136125 20050136090 20050112547 20050106554 20050100877 20050095228 20050084961 20050074435 20050064399 20050064382 20050060779 20050058632 20050048644 20050048036 20050048035 20050048034 20050048033 20050033261 20050025819 20050025755 20050021007 20050019919 20050004663 20050004625 20050002986 20050002915 20040260268 20040254560 20040248293 20040243097 20040241167 20040229205 20040229204 20040209236 20040203089 20040185553 20040185544 20040171148 20040152190 20040137551 20040136974 20040121456 20040106965 20040102415 20040101862 20040096813 20040091859 20040087022 20040067480 20040055030 20040053819 20040053207 20040049016 20040043374 20040043012 20040038997 20040038424 20040033479 20040025519 20040023202 20040022666 20030229143 20030216699 20030215420 20030209479 20030203407 20030199050 20030194693 20030180784 20030175850 20030175818 20030170756 20030166894 20030153079 20030149002 20030134789 20030124099 20030118980 20030114651 20030104504 20030100739 20030100037 20030099965 20030092703 20030049339 20030027924 20030027220 20030017444 20030014089 20030013074 20030003574 20020192197 20020192191 20020188963 20020183869 20020172934 20020169123, 20020165280 20020161313 20020155183 20020150928 20020132225 20020132224 20020123143 20020115706 20020114791 20020098470 20020094570 20020094568 20020090643 20020090365 20020086403 20020086346 20020076445 20020045228 20020045156 20020042597 20020034722 20020034163 20020016625 20020016002 20020015999 20020007215 20020007214 20020007213 20020005206 20010049141 20010049140 20010031492 20050155099 20050032208 20050032218 20050221478 20030235563 20050164380

The PREPARATION of Applicants infusion SC and optionally BM compositions may also employ various differentiation inhibition methods/agents—in order to prevent differentiation and in certain cases proliferation, as may be required. Thus, inhibiting methods and agents may be required in the preparation of the ex-vivo primordial SC proliferation cultures and in the formulation of Applicants' SC/BM composition(s), including Applicants' activated composition. Inhibiting agents are well known in the art. See 6. Qi, X. et al. BMP4 supports self-renewal of embryonic stem cells by inhibiting mitogen-activated protein kinase pathways. Proc Natl Acad Sci USA 101, 6027-6032 (2004); Smith, A. G. et al. Inhibition of pluripotential embryonic stem cell differentiation by purified polypeptides. Nature 336, 688-690 (1988); Williams, R. L. et al. Myeloid leukaemia inhibitory factor maintains the developmental potential of embryonic stem cells. Nature 336, 684-687 (1988); Ying, Q. L., Nichols, J., Chambers, I. and Smith, A. BMP induction of Id proteins suppresses differentiation and sustains embryonic stem cell self-renewal in collaboration with STAT3. Cell 115, 282-292 (2003). Also see for example United States Patent Application 20050153941, Miyabayashi, Tomoyuki; et al., Jul. 14, 2005 which provides for a differentiation inhibiting agent which allows an embryonic stem cell culture in an undifferentiated state without use of any feeder cell, prepared by culturing using a differentiation inhibiting agent of a tetrahydroisoquinoline derivative. Other references disclosing differentiation/proliferation inhibiting agents/methods, include U.S. Pat. Nos. 7,115,267, 6,432,917, 6,022,848, 5,861,483; and, US Patent Publications 20050118713, 20040229350, 20030082803, 20050153941, 20050106725 20050266553 20050079607 Likewise, inducement of proliferation and/or differentiation may also be required in the practice of the invention. The art provides for a number of inducing means. See for example U.S. Pat. Nos. 7,115,267, 5,650,299 and US Patent publications 20050239201 “Methods of inducing differentiation of stem cells into a specific cell lineage.” Also see 20050227353, 20050191744.

### Bone Marrow Activation and Stimulation

The bone marrow is presumed to be more replete with SC's than any other body site, and is represented by all lines-types in proportion to their level of differentiation. Recipient's in-vivo BM will serve as the germination centers or factories of SCs generation in the practice of this invention. BM contains other critical constituents that serve in the process of SC differentiation and specific tissue commitment (i.e. production of progenitor SC's and the like). Applicants' invention utilizes recipient's BM itself to promote and produce, through its natural processes, the needed younger SC's destined to assume residence within specific tissue location.

Direct infusion of Applicant's SCs compositions, including said activated composition inspires recipient's own BM to commence the eventual manufacturing of the desired SC, progenitors and others that will be delivered to tissue sites throughout recipient's body. Thus, toward this goal, the BM is appropriately stimulated and/or activated by one of a variety of disclosed means under this invention to accelerate natural production and deployment of younger DNA aged containing SCs of varying differentiation—SCs from the newly formed seeded BM centers, which have formed as a consequence of the systematic systemic introductions/infusions of the biologically younger DNA containing SCs (of applicants' invention) into the recipient sera/BM.

In the practice of this invention, recipients' BM will ultimately generate sufficient younger DNA containing SCs to populate recipients' entire body. These elevated levels of SCs must be for a reasonable to a reasonably prolong period to effectuate Applicants' tissue chimeras. Elevated SC activity can generally be measured as a function of the elevated stem cell count in recipient's blood stream (a provided herein). Thus, elevated SC count in recipient's blood stream is normally a function of new steam cell generation from recipient's BM—in turn resulting from the practice of Applicants' invention.

Applicants' expect that there may be a gestation period for recipient's own bone marrow to become, itself sufficiently regenerated with the introduction of young DNA containing SCs, before it will in turn generate the increase in necessary SCs for the rest of the body. Thus, the need for a systematic infusion program to commence this indigenous BM activity.

Naturally, immediately after a vascular infusion/injection in the blood stream, one would expect the SC count to be elevated.

However, the indigenous generation of SC's within the bone marrow is a preferred practice of this invention, where said self-generated younger DNA age containing SCs ultimately incorporate into the recipient's end organs and tissues in which the younger DNA containing and, thus eventually differentiated cells (also through promotion and stimulation processes) assume a statistical preponderance defining the recipient's body.

AVOIDANCE OF GRAFT-VERSUS-HOST OR AUTOIMMUNE REACTION is an important element of this invention. Graft-versus-host reaction is an inflammatory response that is unique to allogeneic transplantation. It is an attack of the “new” SCs or bone marrow's immune cells against the recipient's tissues. This can occur even if the donor and recipient are HLA-identical because the immune system can still recognize other differences between their tissues. It is aptly named graft-versus-host reaction because bone marrow transplantation is the only transplant procedure in which the transplanted cells must accept the body rather than the body accepting the new cells.

Acute reaction typically occurs in the first 3 months after an infusion or transplantation and may involve the skin, intestine, or the liver. Chronic graft-versus-host reaction may also develop after allogeneic transplant and is the major source of late complications. In addition to inflammation, chronic graft-versus-host reaction may lead to the development of fibrosis, or scar tissue, similar to scleroderma or other autoimmune reactions and may cause functional disability, and the need for prolonged immunosuppressive therapy. Graft-versus-host reaction may be mediated by T cells when they react to foreign peptides presented on the MHC of the host. Removal of these T cells before donation can lessen the risk of this reaction. As provided herein Applicant's invention is to be practiced in a way to avoid any graft-versus-host reaction.

The use of embryo, placenta and cord SC's substantially mitigates these concerns, as these are normally non-HLA typing.

However, in certain practice an immune reaction is expressly contemplated and methods of suppression will be administered. Various means are contemplated and many are within the skill of the art. See for example, United States Patent Application 20060147428, Sachs; David H., Jul. 6, 2006, which provides methods for restoring or inducing immunocompetence, including the step of introducing donor thymic tissue into the recipient. Autoimmune suppression is thus a contemplated practice and is know in the art. Also see US Patent Publications 20040156834, 20070077201, 20060111316, 20040198762, 20060153819 Other methods include adaptation in-vitro, wherein graft v host mitigation takes place. For example in donor SC and/or BM compositions, where said compositions include recipient-host DNA and adaptive means, prior to injections. A beneficial aspect of the Graft-versus-Host phenomenon is the “GRAFT VERSUS TUMOR EFFECT” or “graft versus leukemia” effect. For example, leukemia patients with chronic graft-versus-host reaction after an allogeneic transplant have a lower risk of leukemia relapse. This is due to a therapeutic immune reaction of the grafted donor lymphocytes, more specifically, the Natural Killer cells, against the reacted bone marrow of the recipient. This lower rate of relapse accounts for the increased success rate of allogeneic transplants compared to transplants from identical twins, and indicates that allogeneic HSCT is a form of immunotherapy. Graft vs tumor is also the major mechanism of benefit of non-myeloablative transplants which do not employ high dose chemotherapy or radiation. As provided herein Applicant's invention may be practiced in order to enhance any graft-versus-tumor effect and therefore will employ appropriate compositions and method that elicit an immune response.

Formation of a dual aged global TRANSIENT TISSUE CHIMERA is one of the most essential elements and objects (if not the most important) of Applicants' invention. It is one of the most distinguishing structures of Applicants invention over the prior art.

Applicants' Transient Tissue Chimera is a distinct in-vivo tissue composition/structure comprised of two genetically distinct types/DNA tissue cell ages. In Applicants' invention a transmogrification of sorts occurs wherein there is a combination of younger aged DNA tissue in a tissue structure together with older DNA containing tissue. The evolvement of this transient tissue chimera eventually produces a replacement of the older tissue with the younger tissue. This transmogrification is a cumulative process and requires systematic infusions (multiple infusions/reinfusions) of Applicants' SCs (and/or SC/BM) compositions, in accordance with the practice of this invention.

Human chimeras were first discovered with the advent of blood typing when it was found that some people had more than one blood type. Most of them proved to be “blood chimeras”—non-identical twins who shared a blood supply in the uterus. Those who were not twins are thought to have blood cells from a twin that died early in gestation. Twin embryos often share a blood supply in the placenta, allowing blood stem cells to pass from one and settle in the bone marrow of the other. About 8% of non-identical twin pairs are chimeras. Many more people are microchimeras and carry smaller numbers of foreign blood cells that may have passed from mother across the placenta, or persist from a blood transfusion. In vitro fertilization (IVF) is also contributing to the number of human chimeras. To improve success rates, two or more embryos are placed in the uterus so women who have IVF have more twin pregnancies than usual. More twins mean more chimeras. In Greek mythology, the Chimera was an awesome fire-breathing monster with the head of a lion, the body of a goat, and the tail of a serpent. The Chimera was killed by the hero Bellerophon mounted, in most versions of the tale, on Pegasus, the winged horse.

In Applicant's invention a “transient tissue chimera” is not an organic original structure, but rather a new structure being introduced in-vivo. This new tissue structure commences as a two part distinctively intertwined differing DNA aged tissue—having two distinct ages and likely two distinct sets of genomes and/or DNA/RNA. The blended/interspersed or dual DNA aged tissue is heterogeneously interwoven within a given tissue structure (muscle, nerve, brain, bone, etc.). The age differential within the chimera eventually diminishes with/after the continuation of periodic (regular/sustained) infusion of the same younger DNA—with the younger DNA tissue of the chimera ultimately dominating, replacing the older DNA tissue. While the entire biological process is still yet known, a global renewal of tissue of all (or virtually all) tissue groups is contemplated.
1. EXAMPLE—An in-vivo transient tissue structure comprised of [at least] two distinct DNA ages, optionally being at least 10 years in age difference.
2. EXAMPLE—A transient tissue structure comprised of [at least] two distinct DNA aged genomes.
3. EXAMPLE—A transient tissue structure comprised of [at least] two distinct DNA aged tissues whereby youngest DNA tissue transmogrifies and replaces the older DNA tissue within said chimera.
4. EXAMPLE—A transient tissue structure comprised of [at least] two distinct DNA ages, whereby said structure incorporates one or more vital organs and the age difference between DNA is at least 10, 20, 30, 40, 50, 60, 70, 80, 90, 100, or more, years.
5. EXAMPLE—A transient tissue structure comprised of [at least] two distinct DNA ages, whereby said structure incorporates the majority of the body's tissues, and the age difference between the DNA is at least 10, 20, 30, 40, 50, 60, 70, 80, 90, 100 years.
6. EXAMPLE—A transient tissue structure comprised of two distinct DNA ages, whereby the younger DNA tissue comprises a plurality of at least 10%, 20%, 30%, 40%, or a majority of 50%, 60%, 70%, 80%, 90%, 95%, or more, of the chimera.
7. EXAMPLE—A transient tissue structure comprised of two distinct DNA ages, whereby the younger DNA tissue transmogrifies the older DNA containing tissue and become 95%, or more [100%], of the chimera.
8. EXAMPLE—A transient tissue structure of the above Examples, wherein after a single, or series of continuing, infusions containing stems cells of said younger DNA, said transient tissue structure of said chimera becomes predominately the age of the younger DNA.
9. EXAMPLE—The Examples above, wherein said chimera is produced after multiple periodic infusion of composition containing a plurality of SCs, and optionally bone marrow, optionally derived from a donor and/or recipient-host (an autologous donor).
10. EXAMPLE—The Example above, wherein after multiple periodic infusion recipient's blood stream enjoys a minimum of a 500% increase in stem cell population (optionally, as measured by use an appropriate marker(s) provided herein or that is otherwise provided in the art).

INJECTION or INTRODUCTION (INFUSION) of Applicants SC and/or BM compositions, as provided herein includes transfusion, infusion, injection, ingestion, transportation, engraftment, inoculation, adsorption, absorption, intercalation, nano-continuous/nano-based or supported transport, physical transport, and all other transport means, whereby Applicants' compositions can be delivered/provided into recipient's body, body tissue, rather orally, via bone marrow, skeletal system, blood stream, vascular department, or any into or by any other system. As provided herein and in Applicants' appended claims term “introduction,” “transportation/transport” and “infusion” are interchangeable terms.

PERIODIC INFUSION REGIMES—As provided herein, an essential element of Applicant's invention is its periodic frequency and active/systematic regimes of infusions, which are required to form the transient tissue chimera. Single and/or haphazard infusions of SCs are not acceptable and do not accomplish Applicants' object of creating a body wide transient tissue chimera, where younger DNA containing tissue eventually replaces older DNA containing tissue.
11. EXAMPLE—A periodic infusion of SC and optionally BM made on an average frequency 1 to 12 times/month over a period ranging from 1 to 12 months/year [ideally continuously for a period of at 2-4 months] with at least two infusions in this 12 month period, and then follow-on infusions of a minimum one infusion per year for a continuous period of time thereafter, or on another frequency sufficient to create a transient tissue chimera characterized as having two distinct DNA tissue ages [optionally, wherein said age difference is at least 10 year, and wherein at least 10%, 25%, or 50% of the chimera tissue is said younger DNA aged tissue].
12. EXAMPLE—A periodic infusion of SC and optionally BM to a recipient under chronological age 20 with said infusion made on an average frequency of 1 to 12 times/month over a cycle period ranging from at least 1 to 3 consecutive months with 1 or more cycles in any 12 to 12 month period, or on another frequency sufficient to create a transient tissue chimera characterized as having tissue of two distinct DNA ages [optionally, wherein said age difference is at least 10 year, wherein at least 10%, 25%, or 50% of the chimera tissue is said younger aged DNA].
13. EXAMPLE—A periodic infusion of SC and optionally BM to a recipient over chronological age 20 with said infusion made on an average frequency of 1 to 12 times/month over a cycle period ranging from at least 1 to 3 consecutive months with 1, or on another frequency sufficient to create a transient tissue chimera characterized as having tissue of [at least] two distinct DNA ages [optionally, wherein said age difference between youngest and oldest is at least 10 years, wherein at least 10%, 25%, 30%, 40%, 50% or more of the transient tissue chimera is said youngest aged DNA].
14. EXAMPLE—A periodic infusion of SC and optionally BM into a recipient at least 30 years of age, wherein said periodic introductions/infusions are made on an average frequency of 1 to 12 times/month over a cycle period ranging for at least 2 consecutive months [preferably 3 or more] within a 12 month period, and on an annual cycle of at least one infusion thereafter for in a period of 1-100 years, or on another frequency sufficient to create a transient tissue chimera characterized as having tissue of [at least] two distinct DNA ages [optionally, wherein said age difference between youngest and oldest is at least 10 years, wherein at least 10%, 25%, 30%, 40%, 50% or more of the transient tissue chimera is said youngest aged DNA].
15. EXAMPLE—A periodic infusion of SC and optionally BM into a recipient on an average frequency of at least once in a single month over a period of 1 to 6 months period or on another frequency sufficient to create a transient tissue chimera characterized as having tissue of [at least] two distinct DNA ages [optionally, wherein said age difference between youngest and oldest is at least 10 years, wherein at least 10%, 25%, 30%, 40%, 50% or more of the transient tissue chimera is said youngest aged DNA].
16. EXAMPLE—A periodic infusion of SC and optionally BM into a recipient over chronological age 20, wherein said introduction/infusion is made on an average frequency of 1 to 12 times/month over a cycle period ranging from at least 1 to 3 consecutive months with 1 or more cycles in any given annual period, or on another frequency sufficient to create a transient tissue chimera characterized as having tissue of [at least] two distinct DNA ages [optionally, wherein said age difference between youngest and oldest is at least 10 years, wherein at least 10%, 25%, 30%, 40%, 50% or more of the transient tissue chimera is said youngest aged DNA].
17. EXAMPLE—A periodic infusion of SC and optionally BM on an average frequency of 1 to 4 times/month for a least one month over a 6 month period, or on another frequency sufficient to create a transient tissue chimera characterized as having tissue of [at least] two distinct DNA ages [optionally, wherein said age difference between youngest and oldest is at least 10 years, wherein at least 10%, 25%, 30%, 40%, 50% or more of the transient tissue chimera is said youngest aged DNA].
18. EXAMPLE—A periodic infusion of SC and optionally BM on an average of once to four times a month for a period of at least two to nine consecutive months in any single calendar year, or on another frequency sufficient to create a transient tissue chimera characterized as having tissue of [at least] two distinct DNA ages [optionally, wherein said age difference between youngest and oldest is at least 10 years, wherein at least 10%, 25%, 30%, 40%, 50% or more of the transient tissue chimera is said youngest aged DNA].
19. EXAMPLE—A periodic infusion of SCs and optionally BM, including the EXAMPLES above, wherein said infusion is made at least once a month in a cycle ranging from 2 to 6 (or more) consecutive months of any given annual period.
20. EXAMPLE—A periodic infusion of SCs and optionally BM, including the EXAMPLES above, wherein said infusion is made at least once a month in a cycle ranging from 2 to 12 consecutive months of any annual period.
21. EXAMPLE—A periodic infusion of SCs and optionally BM on a frequency sufficient to create a transient tissue chimera characterized as having tissue of [at least] two distinct DNA ages [optionally, wherein said age difference between youngest and oldest is at least 10 years, wherein at least 10%, 25%, 30%, 40%, 50% or more of the transient tissue chimera is said youngest aged DNA].
22. EXAMPLE—A periodic infusion regime of SCs and optionally BM wherein a transient tissue chimera is formed containing at least two distinct DNA ages, and wherein after a continuation of infusions containing SCs and optionally BM, said transient tissue chimera enjoys a majority population of tissue being the youngest DNA age.

The art suggests stem cells comprise approximately 0.1-1.0% of the total nucleated cells as measured by the surrogate CD34+ cells. See 20040258673. The formation of a dual aged transient tissue chimera is predicated upon a periodic infusion regime with sufficient dosage that in turn operates to elevate recipients' bone marrow and blood stream stem cell count for a prolonged period of time. While minimal and upper levels and durations are yet unknown, it is expected that there will be some upper limit where the body's spleen and other organs will dispose of excess quantities. However, minimum bone marrow and blood stream quantities of 0.3 to 3.0% of the total nucleated cells are expected minimums. Minimums of 1% to 4%, with about 3% of the total nucleated cells in the bone marrow or blood stream being SCs over a prolonged period of at least 1 to 52/weeks within a year is preferred.
23. EXAMPLE—A periodic infusion regime of SCs and optionally BM, wherein after infusion for a period of at least 3 days to 52 weeks in any given year, recipients average blood stream SC volume is at least .0.2 to 5.0% of total nucleated cells.
24. EXAMPLE—A periodic infusion regime of SCs and optionally BM, wherein after infusion for a period of at least 2 weeks to 52 weeks in any given year, recipients average blood stream SC volume is at least .0.3 to 5.0% of total nucleated cells.
25. EXAMPLE—A periodic infusion regime of SCs and optionally BM, wherein after infusion for a period of at least 4 weeks to 52 weeks in any given year, recipients average blood stream SC volume is at least .0.3 to 5.0% of total nucleated cells.
26. EXAMPLE—A periodic infusion regime of SCs and optionally BM, wherein after infusion for a period of at least 8 weeks to 52 weeks in any given year, recipients average blood stream SC volume is at least .0.3 to 5.0% of total nucleated cells.
27. EXAMPLE—A periodic infusion regime of SCs and optionally BM, wherein after infusion for a period of at least 12 weeks to 52 weeks in any given year, recipients average blood stream SC volume is at least .0.3 to 5.0% of total nucleated cells.
28. EXAMPLE—A periodic infusion regime of SCs and optionally BM, wherein after infusion for a period of at least 8 weeks to 52 weeks in any given year, recipients average blood stream SC volume is at least .1.5 to 5.0% of total nucleated cells
29. EXAMPLE—A periodic infusion regime of SCs and optionally BM, wherein after infusion for a period of at least 12 weeks to 52 weeks in any given year, recipients average blood stream SC volume is at least 1.5 to 5.0% of total nucleated cells.
30. EXAMPLE—A periodic infusion regime of SCs and optionally BM, wherein after infusion for a period of at least 8 weeks to 52 weeks in any given year, recipients average blood stream SC volume is about 2.0%, or greater, of total nucleated cells.

Appropriate markers may be used to measure these volumes. Additional volumes/amounts and ranges are contemplated and provided in the Supplemental Specification.

A principal embodiment of applicants' invention is predicated upon stimulating the inherent production of these younger DNA containing SCs within the body's own bone marrow. By producing a continuous volume of these younger DNA containing SCs itself, the body will naturally generate the means for creating Applicants' global transient tissue chimera. Thus, Applicants' dosages and treatment regime of periodic infusions, which produce the dual age transient tissue chimera, are a critical element of this invention.

IN-VIVO STEM CELL EXPANSION primarily via bone marrow activity, which is accomplished via Applicants' dosages, SC and/or BM compositions, periodic infusions is contemplated. It is the enhanced activity of the bone marrow, in turn producing younger aged DNA containing SCs that fill the recipient's body for a continued period of time, in turn creating the dual age transient tissue chimera that represents the essence of Applicants' invention. Thus, Applicants' invention contemplates various methods of improving SC expansion and proliferation in-vivo after infusion. Applicant will also employ existing method in achieving this goal. See for example United States Patent Application, 20070077201

Employing MARKERS may be appropriate to determine if minimal SC volumes are achieved. Applicants' contemplated practice and appended claims provide for various increases of blood stream populations of stem cells. Different compositions and methods are likely to product different results, different stem cell types, which will populate the blood stream. It is contemplated that various markers will be established as appropriate to determine that these limitations (results) are secured. In the case of hematopoietic progenitor cells, it is known that CD34.sup.+ cells may be a measuring determinate. A highly stem cell concentrated cell composition could see a CD34.sup.+, CD10.sup.−, CD19.sup.− and CD33.sup.−, more particularly in addition CD3.sup.− and CD8.sup.−, preferably in addition Thy-1.sup.+. The CD3.sup.−, 8.sup.−, 10.sup.−, 19.sup.−, 20.sup.− and 33.sup.−. The CD10/19/20 markers are associated with B-cells, CD3/4/8 markers are associated with T-cells, CD14/15/33 cell markers are associated with myeloid cells. The Thy-1 marker is absent on human T-cells. Also, for human CD34.sup.+, rhodamine 123 can divide the cells into high and low subsets. See Spangrude, (1990) Proc. Natl. Acad. Sci. 87, 7433 for a description of the use of rhodamine 123 with mouse stem cells. Preferably the cells are rhodamine low.

However, the blood circulation of embryo totipotent, pluripotent and multipotent SCs (or proliferated derivations) may implicate different markers.

ALLOGENEIC PRACTICE—The allogeneic embodiment of this invention is similar to the autologous practice, except that the original source of the biologically younger stem cell is from a party other than recipient. In this practice an essential element is the conversion or use of a stem cell that is DNA compliant or modifiable to the DNA of recipient. Research suggests a species of pluripotent stem cells from a 3^{rd} party donor may be sufficiently absent DNA characterization to become DNA compliant to that of recipient. See (cite).

DNA compliance under this method, as in the case of the autologous practice, is achieved when the characterization of the transfused stem cell is fully recognized as being biologically younger DNA capable of causing the global integration of stem cells in recipient whereby these biologically younger DNA containing stem cells are integrated into recipient's tissues throughout his/her entire body. This integration forms a transient chimera, which is characterized by the collective tissue composition of the recipient whose chronologically older DNA-containing cells are renewed by the biologically younger DNA-containing infused stem cells, whereby the biologically age differential of resulting cellular structure comprising the resulting transient tissue chimera is 5 years younger or more than the original biological age of the recipient's cellular structure/tissue prior to infusion.

Combination of recipient's DNA with biologically younger pre-transfusion non-HLA typing donor stem cells—in either an in-vitro or in-vivo process and combination process, whereby a younger identical DNA, DNA compliant or otherwise sufficiently similar stem cell to recipient results, such that it is sufficient to effect a global renewal/integration of recipient's cellular tissue with biologically younger DNA as provided under Applicants' infusion means.

The stem cells of this practice include the entire continuum of undifferentiated to differentiated or tissue committed stem cells. In the autologous practice of this invention the collection of a full range of undifferentiated and more differentiated or tissue committed stem cells from the donor for preservation until later transfusion is contemplated. Cell proliferation (augmentation) without new or further differentiating of such is expressly contemplated where the population of stem cells, in the ratio collected, can be preserved.

Laboratory proliferation of differentiated cells—e.g. heart muscle cells, blood cells, or nerve cells, is controlled by the chemical composition of the culture medium, alteration of the surface of the culture dish, or by cellar modify by insertion of specific genes. See Chapters 5-9 and Appendices B and C of the NIH report Stem Cells: Scientific Progress and Future Research Directions.

The essence of this invention is the periodic transfusion of sufficient plurality of younger DNA stem cells compared to the biological age of the recipient, whereby said cells are absorbed into the entire body so as to retard the biological aging process.

Thus, the method contains various means and combination to cause the integration of these biologically younger stem cells to be absorbed and included in the tissues throughout recipient's body, whereby recipient's natural biological aging process is retarded. This is an essential element of the invention.

The dosage levels of the transfused stem cells are expected alone to be sufficient to achieve this result by natural absorption. Thus, the effective concentrations and quantities and frequency of transfused stem cells into the recipient are an essential element of this invention. Furthermore, the collection of bone marrow and its later infusion into recipient, independently of and/or simultaneously with stem cells is expected to also achieve this result. Thus, the infusion of young bone marrow directly or indirectly into recipient's bone marrow is contemplated means. Another would be the infusion of stem cells directly or indirectly into the bone marrow (simultaneously with/or independently of bone marrow infusion). Stimulation of bone marrow activity prior to, during, and after stem cell transfusion is an express embodiment.

Applicants' believe that enhanced bone marrow activity with corresponding production of stem cells and related structure is a critical element in achieving the result of global tissue renewal under this invention. Independent and simultaneous use of signalers, cellular receptors, physiological techniques, drugs, meditation, psychological techniques, electronic stimuli, audio inducers/stimuli (music), and other means are contemplated. Signaling means to encourage specialized adsorption and usage of transfused stem cells.

This means of insuring that said infusion achieves a global integration of biologically younger stem cells within the chronologically older DNA containing cells contemplates facilitation or separate mechanisms, which will yield such results. Contemplated techniques, include:
a. Bone marrow grafting/infusion
   i. Independently or in conjunction with stem cell infusion
b. Systematic infusion technique
c. Periodicy of infusion
d. Frequency/duration infusion therapy
   i. multiple infusions over a relatively short period.
   ii. Prolonged infusion
      1. Continuous multi-hour or multi-day infusion technique.
e. Saturated or massive dosage infusion technique
   i. Dosage sufficient to achieve saturation level for minimal dosage amount and/or infusion period
f. Multi-point transfusion technique
g. Stem cell modification
h. Stem cell compositional modification/tailoring
i. Resonance Therapy
j. Magnetic therapy
k. Electronic
l. Electro-Evaporation. See U.S. Pat. No. 6,972,013,
m. Gene therapy modification. See for example U.S. Pat. No. 7,115,417,
n. Grafting technique
o. Hormone therapy
   i. Growth hormones. See 20060247170, 20060094655
p. Electrical induction or stimulation
q. Audio, music. micro-vibrations or ultrasound based induction or stimulation. See for example: 20040191906
r. Visual based induction or stimulation
s. Light therapy, including photothermal, photochemical and photomodulatory therapy. See for example: U.S. Pat. No. 6,936,044
t. stem cell trafficking, see U.S. Pat. No. 6,814,961
u. Intracellular signaling molecules/technique, see 20060115813
v. Diet
   i. Antigenic sensitive diet (reducing immune sensitivities)
w. Protein therapy
x. Anti-body therapy
y. Protein therapy/injection
z. Stem cell tailoring technique
aa. Stem cell blend proportion technique
   i. Transfusion samples containing certain markers, agents or progenitor stem cells
bb. Tunable Matrixes, see 20070026518

The biological age differentiation between the transfused DNA and bone marrow that recipient receives is also a critical element of this invention. The invention's effectiveness is predicated upon the DNA age of the transfused stem cells and/or bone marrow being younger than the biological age of recipient. The younger the DNA age compared to the biological age of the recipient, the better anticipated tissue renewal is expected.

One embodiment of this invention contemplates a mathematical tailoring of anti-aging effect. Under this embodiment huge differences between the biological age (average biological age) of the transfused DNA and recipient are preferred. However, another embodiment expressly contemplates, especially if significant quantities of stem cell material are available, employing the youngest DNA stem cell material possible for as long as possible.
31. EXAMPLE—An autologous anti-aging method comprising: Periodic collection of whole stem cells and/or bone marrow from donor-recipient from shortly after his/her conception to chronological age 500 years, whereby said collection of stem cells provides a plurality of embryonic and/or adult cells selected from the group consisting of totipotent stem cells, pluripotent stem cells, multipotent stem cells, progenitor stem cells and combination thereof; Providing for long term storage of said donor-recipient stem cells and/or bone marrow in sterile conditions in non-breachable containers; Thawing a portion of said stored stem cells and/or bone marrow after a period of storage; Periodically auto-transfusing or infusing said stem cells and/or bone marrow back into donor-recipient, whereby said infusion results in an integration of biologically younger DNA containing stem cells and/or bone marrow into donor-recipient's bone marrow & tissues throughout his/her body.
32. EXAMPLE—The autologous example above wherein said collections are on average a minimum of once every year, once every two years, once every three years, once every four years for the first 30 years of donor-recipient's life.
33. EXAMPLE—The autologous example above wherein said infusions/transfusions are on average a minimum of once every year, once every two years, once every three years, once every four years, once every five years after chronological age 15, 16, 17, 18, 19, 20, 21, 22, 23, 24, 25, 26, 27, 28, 29, 30 or later of the donor-recipient's life.
34. EXAMPLE—An autologous anti-aging method comprising: Periodic collection of stem cells and/or bone marrow from a donor from after conception; Providing for the long term storage of said donor-recipient stem cells and/or bone marrow in sterile conditions in non-breachable containers; Thawing a portion of said stored stem cells and/or bone marrow after a period of storage; Periodic and regular infusions or infusion of said stem cells and/or bone marrow into the donor starting after age 10 years, whereby said periodic infusions result in an average biological age of the new body comprised of replaced tissues from the donor DNA at least 5 years younger than the actual chronological age of said recipient.
35. EXAMPLE—An anti-aging method comprising: Periodic collection of stem cells and/or bone marrow from DNA related donor from shortly after his/her conception to chronological age 500 years, whereby said collection provides a plurality of embryonic and/or adult cells selected from the group consisting of totipotent stem cells, pluripotent stem cells, multipotent stem cells, progenitor stem cells and combination thereof; Optionally, providing for the long term storage of said donor-recipient stem cells and/or bone marrow in sterile conditions in non-breachable containers; Optionally, thawing a portion of said stored stem cells and/or bone marrow after a period of storage; Periodically transfusing or infusing said stem cells and/or bone marrow into DNA related or compatible recipient, whereby said infusion results in a global integration of biologically younger stem cells into recipient's tissues throughout his/her body.
36. EXAMPLE—An anti-aging method comprising: A periodic collection of stem cells and/or bone marrow from DNA related donor from shortly after his/her conception to chronological age 500 years, whereby said collection provides a plurality of embryonic and/or adult cells selected from the group consisting of totipotent stem cells, pluripotent stem cells, multipotent stem cells, progenitor stem cells and combination thereof; Optionally, providing for the long term storage of said donor-recipient stem cells and/or bone marrow in sterile conditions in non-breachable containers; Optionally, thawing a portion of said stored stem cells and/or bone marrow after a period of storage; Periodically transfusing or infusing said stem cells and/or bone marrow into DNA related or compatible recipient, whereby said infusion results in a global integration of biologically younger stem cells into recipient's tissues throughout his/her body.
37. EXAMPLE—An anti-aging method comprising: The collection of stem cells and/or bone marrow from a biologically suitable donor from after conception providing embryonic and/or adult cells selected from the group consisting of totipotent stem cells, pluripotent stem cells, multipotent stem cells, progenitor stem cells; Optionally, providing for the long term storage of said donor-recipient stem cells and/or bone marrow in sterile conditions in non-breachable containers; Optionally, thawing a portion of said stored stem cells after a period of storage; The adaptation of said stem cells to comport with the DNA structure of recipient; Periodic transfusing of infusing a quantity of said stem cells into recipient, on the proviso that said transfused stem cells are biologically younger than recipient, whereby said infusion results in an integration of said stem cells into recipient's tissues throughout his/her body
38. EXAMPLE—A composition containing DNA in a stem cell carrier and optionally bone marrow, whereby said DNA is delivered in sufficient useable quantity to an autologous donor/recipient to regenerate existing cell structure on a global body basis; wherein said regeneration provides a 5 year differential between the donor/recipient's chronological age and his biological age.
39. EXAMPLE—The anti-aging methods and compositions above, wherein said infusion results in biologically younger DNA containing stem cells being integrated into recipient's tissues throughout his/her entire body causing formation of a transient chimera, characterized by the collective tissue composition of the recipient current chronologically older DNA-containing cells with biologically younger DNA-containing infused stem cells, where the biologically age of the resulting transient tissue chimera is at least 5 years younger than the chronological age of recipient.
40. EXAMPLE—An Allogeneic Anti-Aging method that: a) collects stem cells from cloned embroyonic steam cells, whereby a plurality of said cells are from the group consisting of Totipotent stem cells, Pluripotent stem cells, and Multipotent stem cells; b) increasing the pool of stem cells by cell proliferation means (see United States Patent Application, 20030232430, Cibelli, Jose; et al. Dec. 18, 2003, Methods for making and using reprogrammed human somatic cell nuclei and autologous and isogenic human stem cells); c) providing long term storage of donor-recipient stem cells in sterile and cell protective conditions in non-breachable containers or other acceptable storage or cryogenic conditions; d) retrieving a portion of said stored stem cells after a period of time; e) periodically transfusing said stem cells back into the recipient after such time as said donor's chronological age exceeds the chronological or biological age of the DNA contained in said transfused stem cells by at least 10, 16, 18, 20, 22, 26, 28, 30 years, whereby said infusion results in an integration of biologically younger DNA containing stem cells through-out recipient's entire body; optionally infusing younger DNA containing bone marrow prior to or simultaneously with said infusion of embroyonic stem cells.

Applicants' invention contemplates EMBODIMENTS including those expressed herein and those that are not. For example, the use of a Telomerase as concurrent therapy and/or as co-additive in Applicants' compositions will be considered. Telomerase is known to be an anti-aging enzyme that is important in cellular proliferation, which may be beneficial in combination with other elements of Applicants' invention. Various methods of producing and using telomerase in anti-aging therapy are known. See 20070042962 20060094043 20040253701 20040086518 20030213008 20030099660 20030077758 20030077757 20030059787 20030044953 20030032075 20030009019 20020187471 20020164786 20020155100 20010048917

It is an embodiment of this invention to combine donor stems cells with host/recipient stem cells. It is contemplated that a pre-initiation conferring the host cellular & DNA attributes onto the donor cells IN VITRO or EX-VIVO may be desirable, while at the same time of augmenting the quantities (growing) the collective stem cell mixture.

However, it is important in the practice of proliferation to realize the concept of cellular senescence that cells have a genetically programmed limitation on the number of cellular divisions—whether in vivo, in vitro or ex-vivo. This most likely due to the lack of other dynamic chemical communications/signals/triggers/suppressors that would be available in the body (in vivo), as these cells are in isolation from the total body. Prior research suggests that unaltered cells could only be ‘passed’ (freshly replated for new growth) @ 50 times or more in culture and would progressively reduce in growth or slow in division time, and not being replaced with new progeny—would wither and die (just like the aging process, but in-vitro on an accelerated basis). If harvested freshly cells are added to an already existing cell culture having undergone several generations of divisions—in-vitro cultures tend to last much longer than those that were not ‘freshened’ despite the continuous process of replating (taking a sample and introducing a fresh media). It has been observed that when cells divide and begin to crowd each other—division slows or ceases—so ‘passing’ to new plates (new replication media) to keep the line going becomes critical. It is important to keep cell healthy as cells ‘altered’ by a virus or other defect no longer seemed to have the senescence factor.

In the practice of this invention, Applicants' do not want to ‘alter’ cells or DNA into losing programmed senescence—or risk creating cancer (this is the fundamental process of tumor cells—when they lose their dynamic give and take communicating ability and divide with abandon). Applicants' do however contemplate technique during formation of the transient tissue chimera to ‘freshen’ the replicating cellular DNA with younger DNA that still contains the dynamic feedback and response mechanisms including senescence.

Thus, one embodiment of the augmentation and proliferation process includes ‘fueling’ by new fresh stem cells—either from cord donor source or from saved younger host stem cells whether in vitro, ex-vivo or in vivo.

To sum: younger stems are pooled together—stored and/or grown to some predetermined number of divisions to achieve minimal dosage, and then infused into the recipient.

Another embodiment is the pre-mingling (comingling) of the donor cord and/or other source stem cells, and recipients stem cells (preferably young stem cells) permits transmogrification of the donor stem cells (DNA) to the recipientt's. However, this is not a requisite step—but possibly and enhancing one. Donor (cord) stem cells and younger recipient stem cells can be comingled and replicated (optionally stored) for later plating or direct infusion into the recipient.

Another embodiment includes comingling donor stem cells and younger or concurrent aged recipient DNA stem cells, which are pooled and grown in culture to augment, proliferate, and to preconfer host/recipient attributes through several divisions—but not so many as to approach cellular senescence. This would permit direct infustion. This would permit bypassing the storage step and/or permit storing the mixture for later use. Thus, embodiment would relying upon the donor's stem cell DNA for revitalization of the recipient/host's body—but the recipient's cell contribution would be ‘priming’ the donor-cord DNA—thus enhancing or transmogrifying it to the host/recipient's attributes prior to infusion—promoting greater acceptance and uptake. Applicants' preferred practice of employing pluripotent stem cells, which are typically non-HLA typing, reduces or eliminates immune sensitivities.

The prior embodiment contemplates donor-cord stems/DNA admixing these with any type, age host tissues—but would be directing a specificity of cellular differentiation—which really is the mainstay of the research going on today in stem cell and tissue repair research. I was trying to stay away from getting this specific on tissues—as our initial goal was to renew the ‘entire’ body.

However, ovaries contain a preset # of ova, which are in a miotic stasis (frozen in the DNA division state and are haploids—23 chromosomes). I don't what affect infusions of young revitalizing DNA would have on this. The outer covering of the ovary (containing full complement DNA—46 chromosomes) is dynamic and dividing and would be subject to renewal. In order for tissues to renew—they have to actively divide. Thus, perhaps there would be no affect on the ova-follicules—which would be a problem for aging women—in that these are central to the stimulation and production of critical hormones—the estrogens etc. which influence the thyroid and others. So, on one hand you could have some tissue renewal but incomplete rejuvenation because we cannot create new ova.

While it may be less of a consideration in men—same with spermatogenesis. However, these are NOT preset in numbers in embryogenesis like ova and are an ongoing production factory. This process is essential for the production of testosterone and other factors for male youthfulness.

While ‘renewal’ of global tissues in women occurs (with the exception of the ovaries estrogen factory), it would be likely that a supply exogenous estrogen to complement the rejuvenating process would be required.

In the case of a non-autologous or allogeneic practice, the use of human embryonic stem cells are a preferred practice. They are easily accessible for controlled and specific genetic manipulation. When this facility is combined with their rapid growth, remarkable stability, and ability to mature in vitro into multiple cell types of the body, human embryonic stem cells are an attractive tools. Pathways for human embryonic stem cells in non-autologous practice include genetically manipulated embroyonic cells to introduce appropriate genetic information. This genetic information may either be active or awaiting later activation, once the modified embryonic stem cell has differentiated into the desired cell type.

Embryonic stem cells can be additionally beneficial since these cells can be differentiated in vitro into many cell types, including presumably tissue-specific stem cells, they may provide a constant in vitro source of cellular material. Thus, “adult” stem cells derived from embryonic stem cells may be utilized to optimize protocols for propagation and genetic manipulation techniques for purposes of acquiring optimal cellular material.

The practice of this invention contemplates genetic manipulation of stem cells. This practice is contemplated in the non-autologous portion of the invention. For this purpose genes may be introduced into cells by transfection or transduction. It is contemplated that transfection utilizing chemical or physical methods to introduce new genes into cells would be employed. Usually, small molecules, such as liposomes, as well as other cationic-lipid based particles are employed to facilitate the entry of DNA encoding the gene of interest into the cells. Brief electric shocks are additionally used to facilitate DNA entry into living cells. All of these techniques may be applied to various stem cells of this invention, including human embryonic stem cells. Recipient DNA may disappear after days or weeks, and in rare cases, integrates randomly into host chromosomal DNA. In vitro drug selection strategies allow the isolation and expansion of cells that are stably transfected, as long as they significantly express the newly introduced recipient's gene. Transduction utilizes viral vectors for DNA transfer. Viruses, by nature, introduce DNA or RNA into cells very efficiently. Engineered viruses can be used to introduce almost any genetic information into cells. However, there are usually limitations in the size of the introduced gene. Additionally, some viruses (particularly retroviruses) only infect dividing cells effectively, whereas others (lentiviruses) do not require actively dividing cells. In most cases, the genetic information carried by the viral vector is stably integrated into the host cell genome (the total complement of chromosomes in the cell). In this practice an important parameter that must be carefully monitored is the random integration into the host genome, since this process can induce mutations that lead to malignant transformation or serious gene dysfunction. However, several copies of the recipient's gene may also be integrated into the genome, helping to bypass positional effects and gene silencing. Positional effects are caused by certain areas within the genome and directly influence the activity of the introduced gene. Gene silencing refers to the phenomenon whereby over time, most artificially introduced active genes are turned off by the host cell, a mechanism that is not currently well understood. In these cases, integration of several copies may help to achieve stable gene expression, since a subset of the introduced genes may integrate into favorable sites. In the past, gene silencing and positional effects were a particular problem in mouse hematopoietic stem cells. These problems led to the optimization of retroviral and lentiviral vector systems by the addition of genetic control elements (referred to as chromatin domain insulators and scaffold/matrix attachment regions) into the vectors, resulting in more robust expression in differentiating cell systems, including human embryonic stem cells. See USE OF GENETICALLY MODIFIED STEM CELLS IN EXPERIMENTAL GENE THERAPIES, by Thomas P. Zwaka Center for Cell and Gene Therapy & Department of Molecular and Cellular Biology, Baylor College of Medicine, Houston, Tex. 77030, “Regenerative Medicine 2006,” http://stemcells.nih.gov/info/scireport/2006report.htm. Thus, this or other means are contemplated in the genetic adoption of stem cells in the practice of this invention.

The invention contemplates means for the proliferation/enhancement of stem cell generation within the donor, himself, prior to collection [CITE Existing Art References]

Another embodiment is the proliferation of stem cells, including embroyonic stem cells from the donor. Cell proliferation without differentiating methods/techniques are contemplated. Applicants' have found the presence of a protein called Oct-4, which help characterize undifferentiated cells typically make is an important marker that can be used in the practice of the invention to confirm that cell differentiation is not occurring.

Thus, Applicants' invention expressly contemplates stem cell proliferation, whereby a starting population of stem cells after several months in the laboratory can yield millions of cells. If the resulting cells are unspecialized, like a preferred parent stem cell, said cells are capable of long-term self-renewal. This is an express object of this invention regarding stem cell growth after collection under this invention.

Said method contemplates growing cells in the laboratory known as cell culture. Human embryonic and other stem cells are isolated by transferring the inner cell mass into a plastic laboratory culture dish that contains a nutrient broth known as culture medium. The cells divide and spread over the surface of the dish. The inner surface of the culture dish is typically coated with embryonic skin cells that have been treated so they will not divide. This coating layer of cells a feeder layer give cells a sticky surface to which they can attach. Also, the feeder cells release nutrients into the culture medium. Recently, scientists have devised alternative ways of growing embryonic stem cells. Over the course of several days, the cells of the inner cell mass proliferate and begin to crowd the culture dish. When this occurs, they are removed gently and plated into several fresh culture dishes. The process of replating the cells is repeated many times and for many months, and is called subculturing. After six months or more, an original population of 30 cells of the inner cell mass can yield millions of embryonic stem cells. Embryonic stem cells that have proliferated in cell culture for six or more months do so under this embodiment without differentiating, and are genetically normal to the originally collected embryonic stem cells. (Cite). Other methods exist for progenitor cell proliferation including United States Patent Application, 20060110368, Prosper Cardoso; Felipe; et al., May 25, 2006, “Medium for culturing autologous human progenitor stem cells and applications thereof.”

These contemplated techniques can incorporate means of preserving the ratio of collected stem cell types/species or one to increasing non-differentiated portion of stem cell types, for example to include a higher population of undifferentiated stem cell types. Examples of such techniques would include: _[CITE Existing Art References].

These proliferation methods may be employed immediately after collection or at any point in the process, up to immediately to transfusion into the recipient. Applicants' preferred practice is immediately after collection, assuming techniques are adequately developed. Applicants' practice contemplates embryonic stem cell collection of the donor-recipient or of a live DNA related donor, for example including_[CITE Existing Art References] “Regenerative Medicine 2006,” is a report/reference describing advances made since 2001 and outlines the expectations for future developments in stem cell research, including stem cell biology, using cells from embryos, fetal tissue, and adult tissues. See http://stemcells.nih.gov/info/scireport/2006report.htm. This reference is incorporated herein in its entirety by reference.

Harvesting of stem cells in adults is usually obtained from the peripheral circulation, however a surgical bone marrow method can be used. The donor may receive a stem cell inducing agent (such as Neupogen—G-CSF) a few days before collecting the stem cells. These inducing agents force the stem cells from the bone marrow into the general circulation. Typically it takes 1 day to collect the necessary number of stem cells from a healthy donor. However, in early infancy or youth this period may be extended.

The practice of this invention anticipates means to keep stem cells within their unspecialized mode for purposes of their self renewal, including for a period of years; and identifying (and improving upon) the signals that upon or after transfusion will permit their optimal conversion into specialized cells.

The use of these and signal means induced by certain physiologic or other conditions can induce transfused unspecialized stem cells to become specialized cells with special functions of recipient, such as becoming the beating cells of the heart muscle or the insulin-producing cells of a pancreas.

Thus, it is contemplated that signaling means will be employed to encourage generalized adsorption and usage of transfused stem cells throughout recipient after transfusion. In certain circumstances this will be used to encourage specialized adsorption and usage of transfused stem cells.

The invention contemplates a number of different storage means. These are generally known in the art [CITE Existing Art References, included by reference]. The long term preservation of the healthiest stem cells and bond marrow cultures/samples possible with appropriate identification and tagging are essential in the practice of this invention.

The invention also contemplates the usage of immune suppression, as may be required. Immune suppression in transfusion and grafting of blood and bone marrow is generally known in the prior art and existing art methods are contemplated in the practice of this invention. See United States Patent Application, 20050191309, Kakkis, Emil D, et al.; Sep. 1, 2005,” Induction of antigen specific immunologic tolerance.”

As contemplated in this invention, limitation, structure and enablement of autologous anti-aging and allogeneic anti-aging methods shall be free transferable. For example the infusion frequency, proliferation of the autologous method shall be applicable to the allogeneic methods.

### EXAMPLE

A process or method employing: a) periodicity of infusions offset by the deliberate and regular periodic collections of a person's stem cells, under a protocol that would follow a staggered or stair-stepped ascending schedule—the earliest collected stem cells are utilized first; b) where those collected in the early years (such as in the first decade) would be used for the first infusions in the 1^{st}, 2^{nd}, 3^{rd}, 4^{th} and later decades (depending on supply quantities), and where stem cells collected in the 1^{st}, 2^{nd}, 3^{rd}, 4^{th} and later decades would be used in the 5^{th} and 6^{th} decades, and so on—with obvious adjustment as time, supply and need would dictate.

### EXAMPLE

An anti-aging method or process: a) tanking periodic collection of stem cells from a donor from his birth to his age 500 years or less (until some determinate period prior to this death); b) sorting and concentrating these stem cells in a collection of blood sera contents, such that they would represent a plurality of blood product constituents for use at a future date; c) storing the donor-recipient stem cells in sterile conditions in non-breachable containers under sub-zero temperature sufficient to insure future use; d) thawing a minor portion of said stored stem cells after a period of 1 to 500 years, depending upon the protocol and program of the recipient; e)

then making periodic auto-transfusions of the stored stem cells, which would be thawed for use, back into same donor (recipient) starting after a responsible age (likely to be after donor's chronological age 10 to 30 years); f) conducting this protocol in such a manner that the infusion of stem cell would result in an integration of biologically younger stem cells into recipient's tissues throughout his/her entire body

It is worth bearing in mind that each successive decade of life will actually be characterized by a younger specimen, resulting from prior infusions of younger stem cells that have consequently replaced the older tissue cells. New supplies of younger stem cells will be produced in the younger person to be used and stored for later use despite their chronological age.

In actuality, the use of Applicants' invention may be elected at any point in a person's life—realizing that optimal benefit of extending youth is achieved using the youngest possible DNA-containing stem cells—(with at least an age differential of 10 years and ideally obtained in the first decade of life). This is the biggest challenge, however—obtaining adequate quantities of stem cells during one's 1^{st} decade to survive years of storage for use later in life. While focus on harvesting stem cells after conception is contemplated in the practice of this invention, harvesting in later stages is also expressly contemplated. Later stages of embryonic development, prior to and after child birth, during very early childhood through infancy are emphasized. Regular collections should continue, throughout the span of a person's life, in order to assure supplies later on, together with continuous periodic infusions to enjoy optimal anti-aging benefit.

Controlled-rate freezing with temperature curve monitoring is required. Until required for infusion, stem cells products may be stored in the vapor phase of liquid nitrogen. Storage period is weeks to years, stem cell products must be adequately preserved for extended periods after cryopreservation.

After thawing, stem cell activity is checked for viability. Because granulocytes do not survive cryopreservation, loss of this cell fraction from the collection is expected. To allow survival during freezing and thawing, cells may be optionally placed in a medium containing 7.5-10% dimethyl sulfoxide (or a better medium as may be discerned). Because cells lose viability over a short period in a dimethly sulfoxide medium, infusing the cells immediately after thawing is important embodiment of this invention.

Periodic and regular collection makes it possible to store the necessary minimum amounts of autologous whole stem cell (the autologous DNA carrier) to practice this invention, and is an essential element.

As contemplated herein periodic collection may begin after conception, in utero or at birth (umbilical cord) and continue until prior to the death of the individual. It is anticipated that this invention will be practiced over a recipient's normal life span, which therein will range from birth to age 50, 100, 150, 200, 300, 400, 500, or more years. The ultimate success of the invention has yet to be determined. It is conceivable human life could be extended to age 1000 years.

Periodic stem cell collection will depend upon the specific program results desired, age at which donor starts the regime and other factors. However, expected collections will start at birth and last a life time. The contemplated collection of stem cells is by harvesting stem cells, through sterile mass blood collection and/or cellular dialysis or continuous circulating cell screening techniques, or others contemplated.

It is fully expected that processes/devices and/or methods, including protector, promoter, enhancing, inducing, suppressing, and/or inhibiting, and/or other agents, known and/or which will be developed specific for: 1) harvesting of targeted stem cells from the vascular compartment, bone marrow, and the like, 2) screening and concentration, and the like, and 3) the stem cells subsequent placement back into donor/recipient's tissues at later date, will be employed.

It is contemplated that these processes/devices and/or methods may or may not be concurrently used during collection and/or infusion, and may be used at some time before and/or after said collection and/or infusion.

These processes/devices and/or methods are incorporated into the claims of this invention.

The continuous collection of stem cells is of paramount importance. It is desirable under this invention to obtain sufficient quantities with a goal of no less than 50 to 500 grams per year, more preferably 100 to 2000 grams. Lesser amounts of 1 to 20 grams per year would also be acceptable. Ideally, collection will need to target as much as possible in the earliest years.

A preferred collection period would include collecting 5 grams to 1000 grams of packed stem cells on a frequently of 1 to every 6 months. Another desirable periodic collection frequency includes at least once every 12 months. Other frequencies include once every 1 to 5 years. Yet another example would be collections of no less than once a year for a donor's age 1 & 2, twice a year for ages 3 to 5, three times a year ages 6 through 15, four times a year 16 years or older. Other periods/frequencies are contemplated. Even so, periodic collection, as contemplated in the claims hereto, include periods that are irregular in length and random in nature.

Typically it takes 3 to 5 days to collect the necessary number of stem cells—collection targets of about 5 million expressing cells—per kilogram of the donor's weight—is the goal, but as few as 2 million cells would be acceptable. Collection quantities less than this number may be expanded or augmented through replication or culture techniques.

Thus, a principal embodiment of this invention is dedicated periodic collection, so that there exists a major amount of youthful stem cells stored by an early age, sufficient to accomplish the life time regime of this invention.

An objective of this invention is to have collected at least 0.01 to 3 kilograms of concentrated stem cell material by a donor's chronological age of 20 to 30 years. Acceptable amounts would be 0.02 to 0.5 kilograms. Similar amounts at chronological age of 30 to 40 years would be acceptable. Concentrated stem cell material of at least 0.2 to 1.0 kilograms would however be more desirable. Concentrations ranging from about 1 kilogram to 6 kilograms and from 1.5 kilograms to 5 kilograms by age 30 would be more desirable. Concentration ranges outside these ranges are expressly contemplated in the practice of this invention.

The invention further distinguishes in that it requires a life-long commitment of the recipient and is for the sole use of the donor-recipient.

The current art employs the technique of auto-transfusions as on an as needed basis for completely differentiated whole blood, red blood cells, immune cells, blood clotting factors & clotting cells for transfusions, in response to emergency, life threatening incidents or anticipated surgeries for patients with rare blood types, such as AB (−). Collection, storage and intended use of blood is sporadic and not conducted over a person's lifetime. The intended product of collection are red blood cells predominately with a smaller percentage of white cells and blood clotting components, as is the case industry wide in blood donation.

Applicants' invention is distinguished, because its autologous blood components are concentrated to reflect a plurality of stem cells for an infusion of younger DNA into the human body for global uptake throughout the body's tissues and/or incorporation into bone marrow stem cell production centers. Thus, it is a completely different product than disclosed in the prior art.

### EXAMPLE

A composition containing DNA in a stem cell carrier, where the DNA can be delivered in sufficient useable quantity to a recipient to regenerate/replace his entire existing cellular tissues on a global body basis—by means of the stem cells differentiating into respective specific tissues and these newly differentiated tissues replicating rates out-pacing the older tissues' replicating rates.

### EXAMPLE

A composition containing DNA in a stem cell carrier, where the DNA is supplied by a mixture of stem cells wherein 5% to 98% are embryonic undifferentiated stem cells and the balance are a combination of adult pluripotent, multipotent, progenitor stem cells and combination, where the DNA is delivered in sufficient useable quantity and means to a recipient to regenerate/replace (over time) his existing cellular tissues/structure on a global body basis.

The above composition is collected according to a predetermined life-time regular regimen with an emphasis on the person's 1^{st} and 2^{nd} decades, with stem cell auto-transfusions performed on a predetermined periodicity throughout a person's life, distinguished from being transfused on a stand-by basis, for example, in response to an emergency, anticipated surgery, or specific healthcare desire.

It is further distinguished because it does not target site specific tissue repairs or regeneration. Instead it is an anti-aging mechanism that performs by replacing older tissue cells throughout the “entire” body with cells containing younger DNA.

Applicant's invention employs a technique whereby ‘whole’ stem cells are globally ‘infused’ via the blood stream, disseminating throughout the whole of the body, rather than site specific injections. The invention, thus, capitalizes on the body's natural design and physiologic mechanisms, which permit the receiving and lodging of the newly introduced stem cells into the tissue; integration, feedback and stimulation that results in differentiation and maturation of the stem cells into the adopted resident tissues. It distinguishes in that no other interventions, chemically or mechanically are performed or induced to affect this result, unlike the current art. And the current art exploring anti-aging modalities and technology employs no similar or obvious modal.

The prior art also utilizes stem cells or their components for the purpose of growing or generating tissues in a laboratory setting for later use in repairing or regenerating specific target tissues within the body. Other prior art focuses on specific techniques exploring sub-components of stem cells and their genetic material in an attempt to control the determinants that regulate growth and cellular behavior.

Instant invention intervenes with the aging process and operates to replace damaged tissue globally, through the use of stem cells and/or their components. It simply recruits the body's own inherent and complex processes to reset its biological clock, through saving its own natural resources (younger stem cells) without complex interference to natural body processes or regulation mechanisms; and re-introduces these said resources to the whole body, through a simply infusion into the blood stream. Claimed invention globally disseminates stem cells that carry younger autologous DNA throughout the entire body. Infused younger stems will migrate directly to specific tissues and will also populate bone marrow stem cell production centers, replacing (out-replicating) older resident stem cells and eventually “replace” the body's existing older tissue cells.

The body does not regress, but is transformed to a younger more vital stage. Acquired attributes would be transferred by the body's inherent redundancies in biological systems. For example, we know that memory and other ‘information’ is transferred to new cells from older ones. Thus, the claimed invention extends youth.

There will be eventual genetic fade. That is, each successive collection point will yield stem cells a little bit older than the proceeding. Thus, a person will still age, but theoretically much more slowly. However, the person's biological age and chronological age will widen. The biological age will advance, but at a slower rate than the person's actual chronological age. It will take much longer to reach the end of the reverse moving platform. But, it will eventually be reached.

Extending youth is to be distinguished from extending longevity—as this is occurring, through enhancements in medical services and technology; public health and preventive medicine via vaccines, medications, early detection and intervention of diseases, enhanced nutrition, and reduced exposure to environmental insults etc., healthful living choices and habits; and through improved environmental engineering.

Applicant's invention posses no risk to the individual from rejection reaction, nor requires use of medications to prevent rejection reactions, delicate or risky invasive procedures or toxic effects, which is a distinguishing feature.

The following is excluded from risk exception, such as idiosyncratic reactions or anaphylaxis to any carrier solution or residue, equipment or its materials used to collect or re-infuse the stem cells or to unforeseen events due to poor technique, sanitation, inventory error or other errors—as its potential occurrence is not unique.

The economics of longer life-spans would be offset by cost-shifting of expensive sick care consumptive costs and the long-term financial and social commitment of sustaining lives suffering with chronic illnesses, as people would live longer—healthier!

Essential elements/structure of claims/examples within this description are intended to interchangeable to between various claims/examples. For example, critical elements contained in a composition's claim/example that would constitute or represent a logical critical limitation of a different example or composition (or method), where would be included if such inclusion be logically. The disclosure of this invention intends this interchangeability of critical feature.

The present invention is not to be limited in scope by the specific embodiments described herein. Indeed, various modifications of the invention are contemplated in addition to those described herein, after further consideration of the references, and/or which will become apparent to those skilled in the art from the foregoing description. The skill of the art is changing rapidly and this invention anticipates certain improvements in the collection, proliferation, differentiation, genetic engineering, preservation of SCs, injection methods, and all other aspects of this invention—wherein for example, when improved methods of collection, proliferation and other techniques of employing pluripotent SCs are developed. New improved signaling, genetic engineering, and all other improved methods/compositions in the practice of this invention are herein expressly contemplated in this invention. Such improvements and means, if it not now expressly provided, are intended to fall within the scope of the appended claims.

All references cited herein (including cited patents, published patent applications, publications, references below, AND the references contained in these references) are FULLY incorporated herein by reference—in their entirety and for all purposes to the same extent as if each individual publication, patent or patent application, reference and references within the cited reference were specifically and individually indicated to be incorporated by reference in its entirety for all purposes in this specification. Said incorporated references are intended to provide background, state of the art, direction, and practice suggestions, and are to read in conjunction with this disclosure/specification in an entirety. Thus, this disclosure and appended claims may be modified as required to reflect the provided references.

The citation of any publication is for its disclosure prior to the filing date and should not be construed as an admission that the present invention is not entitled to antedate such publication by virtue of prior invention.

Applicant's autologous practice is predicated upon:
Periodic collection of stem cell material from a donor, whereby the mass of all collected samples has an average biological age ranging from after conception, at birth and to 70 years +/− of age, with preferred biological age ranging from birth to under 50, and a more preferred range from after conception, birth and to under age 30.
Concentration/sorting this stem cell material such that it can be effectively stored for very extended times, absent appreciable aging, and then later reused as a live organic medium. Sorting into primordial SC components and then proliferating said stem cells.
Storing the stem cell medium, whereby said samples do not appreciably age and can be infused into a mammalian being up to decades or centuries later.
Periodic infusion into the donor after achieving a minimum age on a global basis, anticipated to be over their entire lifetime, wherein
The continuation of this periodic stem cell collection and stem cell infusion regime results in an average chronological and/or biological age difference of infused DNA (that is appreciably—at least 5 years younger) compared the chronological age of the donor.

Mammalian cells are pre-programmed, through their genome (DNA mechanism) so as to undergo a finite number of cellular replications—divisions. As the individual cells (which define the total tissue structure) progress along their continuum of number of cellular divisions, their rate of replication slows. Eventually, signs of aging become apparent, as damaged and worn tissue cells are not timely replaced. Cells become worn, exhausted, damaged from environmental insults, and deprived of necessary co-factors and interactive influences from other failing bodily endocrine and physiologic functions.

While research efforts are and have been underway to intervene in the aging process, none propose to simply replace the entire body's cells with younger, fresh cells through a self-adaptive (autologous) grafting process, employing the very body and DNA of the recipient.

Human stems cells contain the body's complete genetic information, factors, determinants and capacity for generation of differentiated tissues throughout the entire body of a person into specific end-organ and support tissues such as bone, nerves, muscle, heart, liver, brain, endocrine organs, skin etc. Human stems cell can be collected, stored and re-infused at a later date into the bloodstream of the same person from which they were harvested at an earlier time in his/her life. They will disseminate throughout the entire body, lodging in all tissues. Interacting with factors and determinants specific to differentiated ‘resident’ tissue cells (already evolved to a specific and distinguishable type of tissue such as bone or muscle, etc.), the stem cells will be induced through interactions with the adopted tissues to ‘differentiate’ or evolved into the respective distinguishable tissues in which they find residence forming a graft and transient chimera of older and younger DNA-containing cells.

Possessing younger DNA, vital enzymes and co-factors, these younger stem cells will divide at faster rates than their adopted perspective resident mature host tissue cells and then mature into new and younger host cells, “replacing” the older resident cells, eventually resulting in an essentially homogeneous cellular composition with respect to the age of the cells' DNA (thus establishing a younger total body).

Infusion with sufficient quantities of vital stem cells over regular, adequate and periodic time-frames would result in successive, but eventually diminishing replacement of aging tissue. Because, it is not known what tissues may have an inherent resistance to ‘replacement’; it is not known what quantities of stem cells would actually be needed and what time period it would take for total tissue replacement with the younger newly differentiated cells; and while it might be presumed based upon natural statistical truths, it is not known if there is a dose-time response curve (more stem cells infused—the faster they take over by replacing the older tissue cells). In addition, one's supply of viable stem cells would eventually be exhausted, as would their and perhaps society's will and resources. An endless supply of one's younger stem cells could not be obtained to be indefinitely accessed by a person later in his/her life.

Prior art research focuses primarily on utilizing human DNA that is obtained in stem cells harvested from donors other than the intended recipient (non-autologous, i.e. embryonic or fetal stem cells). Still other prior art explores autologous DNA obtained from stem cells collected from the donor in real-time or relatively current to the recipient's age and treatment period. Other art discloses both types involve “injecting” whole stem cells (whether autologous or not) into target tissue to promote repair, such as in spinal cord injury or damaged heart.

It is not known or disclosed in the prior art, whether repair occurs due to unique stimulatory factors inherent to stem cells that are exerted upon the resident differentiated damaged tissues or if repair results from the stem cells differentiating themselves into undamaged tissue. To date, the vast focus of autologous stem cell repair employs enucleating the stem cell and transferring this DNA into enucleated cells of the host's tissue to be repaired. This process of enucleating and transferring is mechanically performed.

The applicants instant invention distinguishes over the prior art because it is predicated upon cells being transferred globally “throughout the entire body,” neither involving injecting cells or their contents or any portion thereof. Nor does it involve a directed transferring of the contents of cells (i.e. DNA) into either tissues or into enucleated tissue cells. This invention distinguishes because it requires pre-planned protocols, pre-positioned resources for an intended recipient prior to or soon thereafter their birth, and deliberate and periodic collection, storing and deliberate, and periodic autologous whole stem cell infusions (auto-transfusions) on a global whole body basis.

### EXAMPLE

A process or method employing: a) periodicity of infusions offset by the deliberate and regular periodic collections of a person's stem cells, under a protocol that would follow a staggered or stair-stepped ascending schedule—the earliest collected stem cells are utilized first; b) where those collected in the early years (such as in the first decade) would be used for the first infusions in the 1^{st}, 2^{nd}, 3^{rd}, 4^{th} and later decades (depending on supply quantities), and where stem cells collected in the I, 2^{nd}, 3^{rd}, 4^{th} and later decades would be used in the 5^{th} and 6^{th} decades, and so on—with obvious adjustment as time, supply and need would dictate.

### EXAMPLE

An anti-aging method or process: a) tanking periodic collection of stem cells from a donor from his birth to his age 500 years or less (until some determinate period prior to this death); b) sorting and concentrating these stem cells in a collection of blood sera contents, such that they would represent a plurality of blood product constituents for use at a future date; c) storing the donor-recipient stem cells in sterile conditions in non-breachable containers under sub-zero temperature sufficient to insure future use; d) thawing a minor portion of said stored stem cells after a period of 1 to 500 years, depending upon the protocol and program of the recipient; e)

then making periodic auto-transfusions of the stored stem cells, which would be thawed for use, back into same donor (recipient) starting after a responsible age (likely to be after donor's chronological age 10 to 30 years); f) conducting this protocol in such a manner that the infusion of stem cell would result in an integration of biologically younger stem cells into recipient's tissues throughout his/her entire body

It is worth bearing in mind that each successive decade of life will actually be characterized by a younger specimen, resulting from prior infusions of younger stem cells that have consequently replaced the older tissue cells. New supplies of younger stem cells will be produced in the younger person to be used and stored for later use despite their chronological age.

In actuality, the use of Applicants' invention may be elected at any point in a person's life—realizing that optimal benefit of extending youth is achieved using the youngest possible DNA-containing stem cells—(with at least an age differential of 10 years and ideally obtained in the first decade of life). This is the biggest challenge, however—obtaining adequate quantities of stem cells during one's 1^{st} decade to survive years of storage for use later in life. While focus on harvesting stem cells during childhood is emphasized, regular collections should continue, throughout the span of a person's life, in order to assure supplies later on and continue the infusions and anti-aging benefit.

Periodic and regular collection makes it possible to store the necessary minimum amounts of autologous whole stem cell (DNA) to practice this invention, and is an essential element.

As contemplated herein periodic collection may begin a birth and continue until prior to the death of the individual. It is anticipated that this invention will practiced over normal life times, which there under will range from birth to age 100, 150, 200, 300, 400, 500, or more years. The ultimate success of the invention has yet to be determined. It is conceivable human life could be extended to age 1000 years.

Periodic stem cell collection will depend upon the specific program results desired, age at which donor starts the regime and other factors. However, expected collections will start at birth and last a life time. The contemplated collection of stem cells is by harvesting stem cells, through sterile mass blood collection and/or cellular dialysis or continuous circulating cell screening techniques, or others contemplated.

It is fully expected that processes/devices and/or methods, including protector, promoter, enhancing, inducing, suppressing, and/or inhibiting, and/or other agents, known and/or which will be developed specific for: 1) harvesting of targeted stem cells from the vascular compartment, bone marrow, and the like, 2) screening and concentration, and the like, and 3) their subsequent placement back into donor/recipient's tissues at later date, will be employed.

It is contemplated that these processes/devices and/or methods may or may not be concurrently used during collection and/or infusion, and may be used at some time before and/or after said collection and/or infusion.

These processes/devices and/or methods are incorporated into the claims of this invention.

The continuous collection of stem cells is of paramount importance. It is desirable under this invention to obtain sufficient quantities with a goal of no less than 50 to 500 grams per year, more preferably 100 to 2000 grams. Lesser amounts of 1 to 20 grams per year would also be acceptable. Ideally, collection will need to target as much as possible in the earliest years.

A preferred collection period would include collecting 5 grams to 1000 grams of packed stem cells on a frequently of 1 to every 6 months. Another desirable periodic collection frequency includes at least once every 12 months. Other frequencies include once every 1 to 5 years. Yet another example would be collections of no less than once a year for a donor's age 1 & 2, twice a year for ages 3 to 5, three times a year ages 6 through 15, four times a year 16 years or older. Other periods/frequencies are contemplated. Even so, periodic collection as contemplated in the claims hereto, include periods that are irregular in length and random in nature.

Thus, a principal embodiment of this invention is dedicated periodic collection, so that there exists a major amount of youthful stem cells stored by an early age, sufficient to accomplish the life time regime of this invention.

An objective of this invention is to have collected at least 0.01 to 3 kilograms of concentrated stem cell material by a donor's chronological age of 20 to 30 years. Acceptable amounts would be 0.02 to 0.5 kilograms. Similar amounts at chronological age of 30 to 40 years would be acceptable. Concentrated stem cell material of at least 0.2 to 1.0 kilograms would however be more desirable. Concentrations ranging from about 1 kilogram to 6 kilograms and from 1.5 kilograms to 5 kilograms by age 30 would be more desirable. Concentration ranges outside these ranges are expressly contemplated in the practice of this invention.

The invention further distinguishes in that it requires a life-long commitment of the recipient and is for the sole use of the donor-recipient.

The current art employs the technique of auto-transfusions on an as needed basis for whole blood or blood product transfusions, in response to emergency and life threatening incidents or anticipated surgeries for patients with rare blood types, such as AB (−). Collection and storage of blood is sporadic and not conducted over a person's lifetime. The intended product of collection are red blood cells predominately with a smaller percentage of white cells and blood clotting components, as is the case industry wide in blood donation.

Applicants' invention is distinguished because its autologous blood components are concentrated to reflect a plurality of stem cells for a global infusion of younger DNA into the human body. Thus, it is a completely different product than disclosed in the prior art.

### EXAMPLE

A composition containing DNA in a stem cell carrier, where the DNA is can be delivered in sufficient useable quantity to an autologous donor/recipient to regenerate his existing cell structure on a global body basis.

The above composition is collected according to a predetermined life-time regular regimen with an emphasis on the person's 1^{st} and 2^{nd} decades, with stem cell auto-transfusions performed on a predetermined periodicity throughout a person's life, distinguished from being transfused on a stand-by basis, for example, in response to an emergency or an anticipated surgery;

It is further distinguished because it does not target site specific tissue repairs or regeneration. Instead it is an anti-aging mechanism that performs by replacing older tissue cells throughout the “entire” body.

Applicant's invention employs a technique whereby ‘whole’ stem cells are globally ‘infused’ via the blood stream, disseminating throughout the whole of the body, rather than site specific injections. The invention, thus, capitalizes on the body's natural design and physiologic mechanisms, which permit the receiving and lodging of the newly introduced stem cells into the tissue; integration, feedback and stimulation that results in differentiation and maturation of the stem cells into the adopted resident tissues. It distinguishes in that no other interventions, chemically or mechanically are performed or induced to affect this result, unlike the current art. And the current art exploring anti-aging modalities and technology employs no similar or obvious modal.

The prior art also utilizes stem cells or their components for the purpose of growing or generating tissues in a laboratory setting for later use in repairing or regenerating specific target tissues within the body. Other prior art focuses on specific techniques exploring sub-components of stem cells and their genetic material in an attempt to control the determinants that regulate growth and cellular behavior.

Instant invention intervenes with the aging process and operates to replace damaged tissue globally, through the use of stem cells and/or their components. It simply recruits the body's own inherent and complex processes to reset its biological clock, through saving its own natural resources (younger stem cells) without complex interference to natural body processes or regulation mechanisms; and re-introduces these said resources to the whole body, through a simply infusion into the blood stream. Claimed invention globally disseminates stem cells that carry younger autologous DNA throughout the entire body that will eventually “replace” the existing older tissue cells.
1. It is an objective of this invention to increase into recipients BM and vascular department “activated” younger DNA containing Primordial SCs at an appreciably higher percentage than they would otherwise exist in recipient within the bone marrow, based upon their chronological/biological age, in light of their sex and weight. For example a 30 year old male weighing approximately 200 lbs could be expected to have some portion of his entire nucleated blood in blood stream (optionally, as measured by CD 34+nucleated cells or other marker) to be SCs, with some population preferably being primordial stem cells, such as pluripotent SCs. Elevated SCs in blood circulation are likely to be most progenitors generated in the bone marrow. Applicants' compositions and methods are intended to increase these total amounts of blood stream circulated SCs by a minimum of 3× of normal amounts for an extended period. Thus, the minimum percentage of primordial SC's added to a recipient's vascular department from Applicants's invention will be 3× the number of nucleated cells (optionally, as measured by CD 34+nucleated cells) being SC's compared to normal. Preferred elevations will be much greater, preferably 10× to 1000×, or more.
2. A method is contemplated involving periodic treatment is according another systematic regime for bone marrow transplantation known in the art, but periodically repeated to achieve minimum SC concentrations.
3. A method is contemplated wherein after a periodic infusion of SCs into recipient, at least 0.3 to 3.0% by volume blood in circulation contains SCs.
4. As contemplated herein an infusion dosage amount or after an infusion has been made, the number of stem cells within recipient's blood stream (vascular department) increases above normal limits. Age appears to be a factor in the volumes of SC circulated in the vascular department. Applicants' invention contemplates a minimum of a 100% in vascular department stem cell circulation. More desirable increases would represent 2× to 5× normal circulation amounts. It is expected that at some upper limit the body will reject the increased population of such SC's.
5. As a function of total number of stem cells to total nucleated stem cells contained in recipient's blood stream, optionally measured by the number of CD+34 cells or other indicative marker, circulated stem cells in the blood stream may range from 0.001 to 40.0%, 0.001 to 30.0%, 0.001 to 20.0%, 0.001 to 15.0%, 0.001 to 12.0%, 0.001 to 10.0%, 0.001 to 9.0%, 0.001 to 8.0%, 0.001 to 7.0%, 0.001 to 6.0%, 0.001 to 5.0% 0.001 to 4.0%, 0.001 to 3.0%, 0.001 to 2.0%, 0.001 to 1.0%, 0.001 to 0.50%, 0.001 to 0.40%, 0.001 to 0.30%, 0.001 to 0.25%, 0.001 to 0.20%, 0.001 to 0.25%, 0.001 to 0.20%, 0.001 to 0.15%, 0.001 to 0.010%, 0.001 to 0.05%, 0.30% to 40.0%, 0.30% to 30.0%, 0.30% to 20.0%, 0.30% to 15.0%, 0.30% to 12.0%, 0.30% to 10.0%, 0.30% to 9.0%, 0.30% to 8.0%, 0.30% to 7.0%, 0.30% to 6.0%, 0.30% to 5.0%, 0.30% to 4.0%, 0.30% to 3.0%, 0.30% to 2.0%, 0.30% to 1.0%, 0.30% to 0.50%, 0.30% to 0.40%, 0.50% to 40.0%, 0.50% to 30.0%, 0.50% to 20.0%, 0.50% to 15.0%, 0.50% to 12.0%, 0.50% to 10.0%, 0.50% to 9.0%, 0.50% to 8.0%, 0.50% to 7.0%, 0.50% to 6.0%, 0.50% to 5.0%, 0.50% to 4.0%, 0.50% to 3.0%, 0.50% to 2.0%, 0.50% to 1.0%, 0.750% to 40.0%, 0.750% to 30.0%, 0.750% to 20.0%, 0.750% to 15.0%, 0.750% to 12.0%, 0.750% to 10.0%, 0.750% to 9.0%, 0.750% to 8.0%, 0.750% to 7.0%, 0.750% to 6.0%, 0.750% to 5.0%, 0.750% to 4.0%, 0.750% to 3.0%, 0.750% to 2.0%, 0.750% to 1.0%, 0.7750% to 40.0%, 0.7750% to 30.0%, 0.7750% to 20.0%, 0.7750% to 15.0%, 0.7750% to 12.0%, 0.7750% to 10.0%, 0.7750% to 9.0%, 0.7750% to 8.0%, 0.7750% to 7.0%, 0.7750% to 6.0%, 0.7750% to 5.0%, 0.7750% to 4.0%, 0.7750% to 3.0%, 0.7750% to 2.0%, 0.7750% to 1.0%, 1.0% to 40.0%, 1.0% to 30.0%, 1.0% to 20.0%, 1.0% to 15.0%, 1.0% to 12.0%, 1.0% to 10.0%, 1.0% to 9.0%, 1.0% to 8.0%, 1.0% to 7.0%, 1.0% to 6.0%, 1.0% to 5.0%, 1.0% to 4.0%, 1.0% to 3.0%, 1.0% to 2.0%, 1.5% to 40.0%, 1.5% to 30.0%, 1.5% to 20.0%, 1.5% to 15.0%, 1.5% to 12.0%, 1.5% to 10.0%, 1.5% to 9.0%, 1.5% to 8.0%, 1.5% to 7.0%, 1.5% to 6.0%, 1.5% to 5.0%, 1.5% to 4.0%, 1.5% to 3.0%, 1.5% to 2.0%, 1.75% to 40.0%, 1.75% to 30.0%, 1.75% to 20.0%, 1.75% to 15.0%, 1.75% to 12.0%, 1.75% to 10.0%, 1.75% to 9.0%, 1.75% to 8.0%, 1.75% to 7.0%, 1.75% to 6.0%, 1.75% to 5.0%, 1.75% to 4.0%, 1.75% to 3.0%, 1.75% to 2.0%, 2.0% to 40.0%, 2.0% to 30.0%, 2.0% to 20.0%, 2.0% to 15.0%, 2.0% to 12.0%, 2.0% to 10.0%, 2.0% to 9.0%, 2.0% to 8.0%, 2.0% to 7.0%, 2.0% to 6.0%, 2.0% to 5.0%, 2.0% to 4.0%, 2.0% to 3.0%, 2.25% to 40.0%, 2.25% to 30.0%, 2.25% to 20.0%, 2.25% to 15.0%, 2.25% to 12.0%, 2.25% to 10.0%, 2.25% to 9.0%, 2.25% to 8.0%, 2.25% to 7.0%, 2.25% to 6.0%, 2.25% to 5.0%, 2.25% to 4.0%, 2.25% to 3.0%, 2.5% to 40.0%, 2.5% to 30.0%, 2.5% to 20.0%, 2.5% to 15.0%, 2.5% to 12.0%, 2.5% to 10.0%, 2.5% to 9.0%, 2.5% to 8.0%, 2.5% to 7.0%, 2.5% to 6.0%, 2.5% to 5.0%, 2.5% to 4.0%, 2.5% to 3.0%, 2.75% to 40.0%, 2.75% to 30.0%, 2.75% to 20.0%, 2.75% to 15.0%, 2.75% to 12.0%, 2.75% to 10.0%, 2.75% to 9.0%, 2.75% to 8.0%, 2.75% to 7.0%, 2.75% to 6.0%, 2.75% to 5.0%, 2.75% to 4.0%, 2.75% to 3.0%, 3.0% to 40.0%, 3.0% to 30.0%, 3.0% to 20.0%, 3.0% to 15.0%, 3.0% to 12.0%, 3.0% to 10.0%, 3.0% to 9.0%, 3.0% to 8.0%, 3.0% to 7.0%, 3.0% to 6.0%, 3.0% to 5.0%, 3.0% to 4.0%, 3.25% to 40.0%, 3.25% to 30.0%, 3.25% to 20.0%, 3.25% to 15.0%, 3.25% to 12.0%, 3.25% to 10.0%, 3.25% to 9.0%, 3.25% to 8.0%, 3.25% to 7.0%, 3.25% to 6.0%, 3.25% to 5.0%, 3.25% to 4.0%, 3.5% to 40.0%, 3.5% to 30.0%, 3.5% to 20.0%, 3.5% to 15.0%, 3.5% to 12.0%, 3.5% to 10.0%, 3.5% to 9.0%, 3.5% to 8.0%, 3.5% to 7.0%, 3.5% to 6.0%, 3.5% to 5.0%, 3.5% to 4.0% of total nucleated cells. Minimal preferred circulation amounts after infusion are 0.01, 0.05, 0.1, 0.15, 0.2, 0.25, 0.30, 0.35, 0.40, 0.45, 0.50, 0.55, 0.60, 0.65, 0.70, 0.75, 0.80, 0.85, 0.90, 0.95, 1.00, 1.01, 1.05, 1.1, 1.15, 1.2, 1.25, 1.30, 1.35, 1.40, 1.45, 1.50, 1.55, 1.60, 1.65, 1.70, 1.75, 1.80, 1.85, 1.90, 1.95, 2.00, 1.01, 1.05, 1.1, 1.15, 1.2, 1.25, 1.30, 1.35, 1.40, 1.45, 1.50, 1.55, 1.60, 1.65, 1.70, 1.75, 1.80, 1.85, 1.90, 1.95, 2.00, 2.01, 2.05, 2.1, 2.15, 2.2, 2.25, 2.30, 2.35, 2.40, 2.45, 2.50, 2.55, 2.60, 2.65, 2.70, 2.75, 2.80, 2.85, 2.90, 2.95, 3.00, 3.01, 3.05, 3.10, 3.15, 3.2, 3.25, 3.30, 3.35, 3.40, 3.45, 3.50, 3.55, 3.60, 3.65, 3.70, 3.75, 3.80, 3.85, 3.90, 3.95, 4.00, 4.01, 4.05, 4.1, 4.15, 4.2, 4.25, 4.30, 4.35, 4.40, 4.45, 4.50, 4.55, 4.60, 4.65, 4.70, 4.75, 4.80, 4.85, 4.90, 4.95, 5.00, 5.01, 5.05, 5.1, 5.15, 5.2, 5.25, 5.30, 5.35, 5.40, 5.45, 5.50, 5.55, 5.60, 5.65, 5.70, 5.75, 5.80, 5.85, 5.90, 5.95, 6.00, 6.01, 6.05, 6.1, 6.15, 6.2, 6.25, 6.30, 6.35, 6.40, 6.45, 6.50, 6.55, 6.60, 6.65, 6.70, 6.75, 6.80, 6.85, 6.90, 6.95, 7.0
6. A method is contemplated in which infusions begin no later than after donor's 1st decade of chronological life, no later than after donor's 2nd decade of chronological life, no later than after donor's 3rd decade of chronological life or no later than after donor's 4th decade of chronological life.
7. The method of the present invention contemplates the difference between resulting average biological age of the recipient's transformed body following infusions of DNA-containing stem cells is at least 5 years younger than the recipient's chronological age, and alternatively, at least 10 years, 20 years, 30 years, 40 years, 50 years, 60 years or 100 years.
8. The anti-aging method of the present invention contemplates collecting stem cells, separating out primordial stem cells, collecting bone marrow, optionally storing said stem cells and bone marrow, augmenting and proliferating said stem cells and bone marrow ex-vivo, periodically infusing into recipient's a DNA compatible composition comprising a:
   a. Fraction of recipient compatible or matched stem cells, where said fraction contains a plurality of primordial stem cells, whereby said steam cells are biologically at least 5 years younger than recipient, and
   b. Fraction of recipient compatible or matched bone marrow
   c. wherein after said infusion the total number of stem cells contained in recipient's blood stream represents at least 0.2% of the total number of nucleated cells contained in recipient's blood stream.
9. Use of younger bone marrow—younger than host-recipient derived from autologous and 3^{rd} party compatible donors is contemplated. Bone Marrow may be extracted from donors under age 5, 10, 15, 20, 25, 30. Bone marrow may also be extracted from host-recipient prior to the infusion. It is contemplated that the use of youthful bone marrow where available will be preferred. To the extent that replication and proliferation technology is available to increase collected marrow, such technologies will be employed.
10. These ranges and other ranges are contemplated as is necessary, on the proviso that dosage together with the frequency of infusions results in the formation of transient chimera tissue, which is biologically younger than the chronological age of recipient. The above infused percentage amount may be measured as a total of recipient's total nucleated cell blood supply [optionally as measured by the number of CD+34 cells or other suitable marker contained in recipient's blood stream, or be measured volumetrically on the basis of total blood contained recipient's body]. Preferred amounts include 0.03% to 0.5%, 0.3 to 0.5%, 0.1 to 1.0%, 0.3 to 1.0%, 1.0 to 4.0%, 2.0 to 3.0%, 2.20% to 3.0%, and about 3.0% of recipient's total nucleated cell blood supply.
   a. The infusions can comprise, alternatively, about 0.010 to 20.0 ounces of stem cells, about 0.2 to 1.0 ounce of stem cells, about 0.2 to 0.6 ounce of stem cells, about 2.0 to 20.0 ounces of stem cells, about 0.5 to 10.0 ounces of stem cells, about 0.5 to 5.0 ounces of stem cells, about 0.1 to 4.0 ounces of stem cells, about 0.1 to 3.0 ounces of stem cells, about 0.1 to 2.0 ounces of stem cells, about 0.10 to 1.0 ounce of stem cells, about 0.10 to 0.8 ounces of stem cells, about 0.2 to 0.6 ounces of stem cells.
11. Volumetrically, single stem cell infusion volumes may range from 1 cc to 5,000 cc's.

Ranges of 1 to 2 cc's, 2 to 8 cc's, 3 to 10 cc's are contemplated. Other amounts would include approximately ¼ to ¾, ⅓ to ½ of the standard volume of a 10 cc hypodermic syringe. Serum carrier materials are expressly contemplated in the delivery of infusions in the practice of this invention.

### EXAMPLE

A composition containing DNA in a stem cell carrier and optionally bone marrow, whereby said DNA is delivered in sufficient useable quantity to an autologous donor/recipient to regenerate existing cell structure on a global body basis.

### EXAMPLE METHODS

An autologous anti-aging method comprising:
   Periodic collection of stem cells and/or bone marrow from a donor from after conception to an adult chronological age,
   Providing for the long term storage of said donor-recipient stem cells and/or bone marrow in sterile conditions in non-breachable containers
   Thawing a portion of said stored stem cells and/or bone marrow after a period of storage
   Periodic and regular infusions or infusion of said stem cells and/or bone marrow into the donor starting after age 10 years, whereby said periodic infusions result in an average biological age of the new body comprised of replaced tissues from the donor DNA at least 5 years younger than the actual chronological age of said recipient.
An anti-aging method comprising:
   Periodic collection of stem cells and/or bone marrow from DNA related donor from shortly after his/her conception to chronological age 500 years, whereby said collection provides a plurality of embryonic and/or adult cells selected from the group consisting of totipotent stem cells, pluripotent stem cells, multipotent stem cells, progenitor stem cells and combination thereof
   Optionally, providing for the long term storage of said donor stem cells and/or bone marrow in sterile conditions in non-breachable containers
   Optionally, thawing a portion of said stored stem cells and/or bone marrow after a period of storage
   Periodically transfusing or infusing said stem cells and/or bone marrow into DNA related or compatible recipient, whereby said infusion results in a global integration of biologically younger stem cells into recipient's tissues throughout his/her body.
An anti-aging method comprising:
   The collection of stem cells and/or bone marrow from a biologically suitable donor from after conception providing embryonic and/or adult cells selected from the group consisting of totipotent stem cells, pluripotent stem cells, multipotent stem cells, progenitor stem cells.
   Optionally, providing for the long term storage of said donor-recipient stem cells and/or bone marrow in sterile conditions in non-breachable containers
   Optionally, thawing a portion of said stored stem cells after a period of storage.
   The adaptation of said stem cells to comport with the DNA structure of recipient.
   Periodic transfusing of infusing a quantity of said stem cells into recipient, on the proviso that said transfused stem cells are biologically younger than recipient, whereby said infusion results in the global integration of said stem cells into recipient's tissues throughout his/her body
In the above example methods, global integration of stem cells results in:
   Biologically younger DNA containing stem cells being integrated into recipient's tissues throughout his/her entire body
   Whereby a transient chimera is formed, characterized by the collective tissue composition of the recipient current chronologically older DNA-containing cells with biologically younger DNA-containing infused stem cells, whereby the biologically age differential of cells comprising the resulting transient tissue chimera is 5 years or greater

Listed below are methodologies contemplated for use in the above example methods either in the alternative or selectively together.
Infusion includes a means for a global integration of said biologically younger stem cells with chronologically older DNA containing cells.
Collection and transfusion includes the collection and transfusion or infusion of stem cells.
Infusion may be by means of a short series of two of more infusions within a period of less than a month.
Infusion may be by means of an extended long duration infusion of a period ranging from 5 minutes to one week.
Infusion may be by means of massive dosage of at least 0.0000000001% 0.000000001% 0.00000001% 0.00000001% 0.00001%, 0.0001% 0.001% 0.01% 0.1%, 1.0%, 10.0% or more of the body weight of recipient.
The methods may employ biologically younger non-HLA typing donor stem cells
The stem cell may pre-transfusion
Collected and transfused stem cells are selected from the group consisting of totipotent, pluripotent, multipotent stem cells and combination thereof.
Transfused stem cells are provided in same relative proportion to those collected.
Transfused stem cells represent a plurality of undifferentiated stem cells.
Transferred stem cells represent a majority of undifferentiated stem cells.
Transferred stem cells are exclusively undifferentiated stem cells.
Stem cells are embryonic and adult stem cells, and combination thereof.
Stem cells are embryonic stem cells or proliferated derivatives thereof.
At any point said stem cells are proliferated in quantity from original quantity collected.
Adaptation of said stem cells is made to comport with the DNA structure of recipient.
Donor stem cells or bone barrow are stored in sub-zero cryogenic temperatures in sterile non-breachable containers with means to preserve said stem cells or marrow indefinitely.
Collection is from harvesting stem cells collection means.
Means of collecting said stem cells is selected from an embryonic, umbilical, bone marrow, sterile mass blood, cellular dialysis, continuous circulating cell technique means and combination.
Collection of said stem cells is by means specific for harvesting targeted cells from the vascular compartment.
Collection of said stem cells is by concentration means specific for harvesting targeted cells from bone marrow.
Concentration of said stem cells, is by means sufficient to result in a plurality of stem cells, which are in combination with blood product constituents.
Collection obtains at least on the order of 10.sup.6 total nucleated cells per Kg. weight of the person in a single collection session.
Each unit comprises a dosage of a fraction of the total nucleated cells required to be transfused or infused.
The units collected or transfused contain equal or different dosages.
The preserving or storage step is independent of tissue or HLA typing of the collected stem cells prior to storing in a stem cell bank.
The preserving or storage step comprises the step of determining from the collected stem cells at least a distinctive property associated with the person prior to storing in a the stem cell bank, so as to provide a means of secured identification to match the collected stem cells with the person at the time of use.
The typing step includes providing indicia with each unit representing information of said at least one distinctive property.
The indicia is embodied in at least one of a label, bar code, magnetic strip, and microchip.
The stem cells are collected during a pre-disease stage in which the person may be diagnosed with a health condition.
Collection and proliferation result in storage of at least 1000 grams of stem cells or material containing a plurality to a majority of said stem cells.
Collection and proliferation results in storage of at least 500 grams of stem or material containing a plurality to a majority of said stem cells.
Collection and proliferation results in storage of at least 250 grams of stem cells or material containing a plurality to a majority of said stem cells.
Collection and proliferation results in storage of at least 50 grams of stem or material containing a plurality to a majority of said stem cells.
An amount is the result of a single collection session or one or more collection sessions and which exists in inventory.
The mass is collected by donor's chronological age of 30, 40 or 50.
Collection is no more frequently than every three months, every six months or every 5 years.
Collection is no less than once a year for a donor chronological age 1 & 2, no less than twice a year for a donor ages 3 to 5, three times a year ages 6 through 15, and annually for ages 16 years or older.
A plurality to a majority of said collection occurs during the first 50 years of donor's chronological life.
A plurality to a majority of collection occurs during the first 10 to 40 years of said donor's chronological life.
Agents increase the quantity, quality and accessibility of stem cells within the circulation.
After said concentration, said stem cells are pooled, packed and batched according to incremental age time frames of collection.
Said incremental time frame of stem cell and/or bone marrow collection is from immediately after conception to birth (umbilical cord), birth to 2 years, 3 to 5 years, 5 to 10 years, 11 to 15 years, and in 5-year increments thereafter.
Collection after conception does not interfere in any way with embroyonic development.
The minimum mass of stem cells collected is equivalent to 10% to 100% of a standard unit of red blood cells (no less than 25 grams) standard in the blood banking industry.
The storing of said donor-recipient stem cells is under sterile conditions in non-breachable containers at temperature below 0° F., −50° F., −200° F., −300° F., or −400° F.
The temperature is a cryogenic freezing temperature sufficient to insure long term storage of said stem cells.
The storage is under liquid nitrogen.
The storage of said stem cells comprises:
   Securely enclosing said stem cells in non breachable containers suspended in bio-compatible nutrient media.
   Labeling, dating & entering into a secure data base (with redundancy and system controls for accuracy and backup in alternate locations)
   Storing said containers under liquid nitrogen.
The storing said stem cells is within a bio-compatible media suspension.
The periodic infusion results in integration of biologically younger stem cells into recipient tissues throughout his/her body at least once every five years, which are comprised of the recipient current chronologically older aged DNA-containing cells.
The infusion results in the creation of a transient tissue chimera—age difference between said infused stem cells (collected and stored years earlier) and recipient's currently aged DNA containing cells is 20 years or greater.
The infusion incorporates harvested stem cells, which have been collected at least 5, 10, 20, 30, 40, or 100 years earlier.
The infusion contains a mixture of stem cells collected over various dates.
The infusion incorporates means for selecting stem cells for maximizing anti-aging response.
The infusion is by means of transfusions or grafting of earlier collected stem cells or bone marrow.
The infusion delivers said stem cells or bone marrow by means of indirect transportation via said donor's blood stream.
The infusion delivers said stem cells or bone marrow by means of direct transportation via direct infusion into recipient's bone marrow.
The infusion is indirect by single or multiple access ports of entry into the blood stream, whereby said infused stem cells find their way to all body tissue sites.
The infusion is coupled with bone marrow infusion.
The stem cells, lodge and become integrated into said donor's cell structure (integrated component).
The integration is by self means.
The infusion of said stem cells throughout said body's tissues are enhanced with respect to delivery, placement and integration within said donor/recipient's tissues, by means of preparing, promoting, and inducing agents.
The infusions begin no later than in said donor's 1^{st}, 2^{nd}, 3^{rd}, 4^{th}, or 5^{th} decade of chronological life.
The infusions are made at intervals ranging from once every month to once every 5 years.
The infusion into said donor is under a periodicity of no less than once every 5-year.
The infusion into said donor is of a period of no less than at 10-year intervals.
The stem cells, which are collected after said donor has reached a chronological age of 40 years or greater, are characterized as being of a biological age no greater than 25 years.
The stem cells, which are collected after said donor has reached a chronological age of 50 years or greater, are characterized as being of a biological age no greater than 35 years.
The stem cells, which are collected after said donor has reached a chronological age of 100 years or greater, are characterized as being of a biological age no greater than 55 years.
The infusion continues until at least chronological age of 100 years.
The stem cell samples are collected within a collection period prior to donor's first infusion, whereby said stem cells have an average chronological age equivalent to their biological DNA age.
The stem cell samples are collected within a collection period prior to donor's infusion, whereby upon infusion said stem cells have an average biological DNA age at least 5 years less than the chronological age of said donor/recipient
The difference between the infused biological DNA stem cell age and the chronological age of the donor/recipient increases, as the chronological age of the donor/recipient increases.
The said stem cells are collected prior to donor's chronological age of 10 years, 20 years, 30 years, 40 years, 50 years or 60 years.
The difference between resulting average biological age of the recipient's transformed body following infusions of DNA-containing stem cells is at least 5 years, 20 years, 30 years, 40 years, 50 years or 60 years younger than the recipient's chronological age.
A composition containing DNA in a stem cell carrier and optionally bone marrow, whereby said DNA is delivered in sufficient useable quantity to an autologous donor/recipient to regenerate existing cell structure on a global body basis.
The composition of wherein said regeneration provides a 5 year differential between the donor/recipient's chronological age and his biological age.
The composition wherein the age difference is no less than 20 years.
The composition wherein concentrated stem cells represent a plurality to a majority of the composition.
The activity resulting from said infusion into said recipient results in global cellar replacement of biologically younger DNA.
The method is allogeneic.
The allogeneic method wherein umbilical cord, embroyonic stem cell are employed.
The allogeneic method wherein embroyonic stem cells are employed taken from cloned embroyonic stem cells
At least 3 billion nucleated stem cells are collected per session.
The periodic collection and infusion methods are pro-active, absent response or in anticipation of any disease.
The harvesting/collection of stem cells comprises making one or more insertions of a syringe means into the donor's hip or pelvic bone to extract bone marrow as said biological specimen containing stem cells.
The stem cells comprise adult hematopietic stem cells.
Introducing a stem cell growth stimulating agent into the donor prior to the harvesting in a manner effective to increase the population of the stem cells in the peripheral blood of the donor before harvesting the specimen.
An anti-aging method comprising of collecting stem cells and/or separating out primordial stem cells selected from the group consisting of totipotent and pluripotent stem cells, optionally storing said stem cells, proliferating said primordial stem cells ex-vivo, infusing a stem cell product comprising a plurality of primordial stem cells into recipient in an amount representing 0.001 to 20.0% of the total nucleated cells contained in recipient's blood stream.
An anti-aging method comprising periodically infusing a product containing a plurality of ex-vivo proliferated primordial stem cells selected from the group consisting of totipotent and pluripotent stem cells and combination, in an amount representing 0.001 to 20.0% of the total nucleated cells contained in recipient's blood stream.
An autologous anti-aging method comprising:
   Periodic collection of whole stem cells from donor-recipient from shortly after his/her conception to chronological age 500 years, whereby said collection of stem cells provides a plurality of embryonic and/or adult cells selected from the group consisting of totipotent stem cells, pluripotent stem cells, multipotent stem cells, progenitor stem cells and combination thereof
   Providing for long term storage of said donor-recipient stem cells and/or bone marrow in sterile conditions in non-breachable containers
   Thawing a portion of said stored stem cells and/or bone marrow after a period of storage
   Proliferating said stem cells
Periodic infusion of stem cells and/or bone into recipient results in an integration of biologically younger DNA containing stem cells and/or bone marrow throughout recipient's bone marrow & body tissues.
A plurality of said infused stem cells are pluripotent.
A majority of all said infused stem cells are pluripotent.
Substantially all said infused stem cells are pluripotent.
The stem cells are ex-vivo derived from an embroyonic stem cell.
The stem cell are ex-vivo derived from an from umbilical cord stem cell
The stem cell is an ex-vivo cell derived from a donor under age 10.
Donor stem cells are matched to recipient's DNA.
Donor stem are absent a HLA typing.

Infusion product contains recipient's own DNA, stem cell and/or derivative
The stem cell is an autogolous stem cell.
The methods above being allogeneic, wherein said infusion product results in recipient experiencing no immune sensitivity/response.
Infusion is vascular, targets cellular renewal/regeneration of recipient's bone marrow, infusion represents 0.1 to 5.0% of the total nucleated cells contained in recipient's blood stream, wherein said infusion represents 0.1 to 1.0% of the total nucleated cells contained in recipient's blood stream, wherein said infusion represents 0.3 to 4.0% of the total nucleated cells contained in recipient's blood stream, wherein said infusion represents 1.0 to 3.0% of the total nucleated cells contained in recipient's blood stream, wherein said infusion represents approximately 2.0% to 3.0% of the total nucleated cells contained in recipient's blood stream, and/or wherein said infusion represents approximately 3.0% of the total nucleated cells contained in recipient's blood stream, wherein said infusion is made on an average frequency 1 to 4 times/month over a period ranging from 2 to 12 months
The recipient is under chronological age 20 and said infusion is made on an average frequency of 1 to 4 times/month over a period ranging from 2 to 9 months
The average frequency is at least once a month over a period of 3 to 6 months
The recipient is over chronological age 20 and said infusion is made on an average frequency of 1 to 3 times/month over a period ranging from 2 to 9 months
The average frequency is once a month over a period of 6 months
The periodic treatment is on average once to four times a month for a period of at least two to nine consecutive months in any single calendar year
At least one or more infusions are repeated within the year following said infusions.
The series of infusions is repeated at least once every other year for recipient's age less than 40.
The recipient is over chronological age 30 and said infusion is made on an average frequency of once a month for the balance of recipient's life.
The series of 2 to 12 consecutive infusions averaging 1 to 4 infusions/month is repeated at least once every 5 years for recipients under age 40.
The recipient is over chronological age 40 and said infusion is made on an average frequency of once a month for the balance of recipient's life.
The infusion product contains DNA in a stem cell carrier and optionally bone marrow, whereby said DNA is delivered in sufficient useable quantity to a recipient to regenerate existing cell structure on a global body basis.
The periodic infusion results in a global integration of biologically younger stem cells into recipient's tissues throughout his/her entire body, characterized by the formation of a transient chimera having a collective biological tissue composition age younger than recipient's chronological age.
The biologically age differential of cells comprising the resultant transient tissue chimera is at least 5 years younger or 10 years younger than the chronological age of recipient.
Periodic collection of stem cells and/or bone marrow from a recipient from the period shortly after his/her conception to chronological age 500 years,
Separating, augmenting, proliferating said stem cells, independently or in combination with allogeneic donor cells under conditions where DNA matching exists
The plurality of said stem cells are pluripotent stem cells.
The recipient's blood contains on average of at least 0.3% to 3.0.0% volume of said pluripotent stem cells on a total nucleated cell basis for at least 72 hours.
The allogeneic method comprises means of converting donor's stem cell's to the DNA characterization of recipient
The stem cells are embroyonic stem cells, cord stem cells and proliferations thereof.
The infusion product is the result of ex-vivo means.
The infusion is by means of engraftment.
The collection and transfusion includes the collection and transfusion or infusion of stem cells.
The infusion is by means of a short series of two of more infusions within a period of less than a month.
The infusion is by means of an extended long duration infusion of a period ranging from 5 minutes to one week.
The infusion is by means of massive dosage of at least 0.0000000001% 0.000000001% 0.00000001% 0.00000001% 0.00001%, 0.0001% 0.001% 0.01% 0.1%, 1.0%, 10.0% or more of the body weight of recipient.
A combination of recipient's DNA with biologically younger non-HLA typing donor stem cells, resulting from an in-vitro or in-vivo process, whereby a younger identical DNA, DNA compliant or an otherwise sufficiently similar stem cell to recipient is produced, such that it is able to effect said global renewal/integration of recipient's cellular tissue with biologically compatible younger DNA.
The method employs biologically younger non-HLA typing donor stem cells
The stem cell is pre-transfusion
The collection and transfusion includes both the collection and transfusion or infusion of bone marrow and stem cells.
The collection and transfusion or infusion includes the collection and infusion of bone marrow.
The transfusion comprises infusing younger bone marrow into recipient's bone marrow
The collected and transfused stem cells are selected from the group consisting of totipotent, pluripotent, multipotent stem cells and combination thereof.
The transfused stem cells are provided in same relative proportion to those collected.
The transfused stem cells represent a plurality of undifferentiated stem cells.
The transferred stem cells represent a majority of undifferentiated stem cells.
The transferred stem cells are exclusively undifferentiated stem cells.
The stem cells are embryonic and adult stem cells, and combination thereof.
The stem cells are embryonic stem cells or proliferated derivatives thereof.
Wherein at any point said stem cells are proliferated in quantity from original quantity collected.
A recipient autoimmune suppression means.
The adaptation of said stem cells is made to comport with the DNA structure of recipient.
The additionally containing a signaling means to encourage generalized adsorption and usage of transfused stem cells.
The undifferentiated stem cell is characterized by containing an Oct-4 protein.
The collected stem cells are from the umbilical cord
The collected stem cells are concentrated, augmented or proliferated
The collected stem cells are sorted, concentrated, augmented or proliferated, and combination.
The stem cells in their collected sera contents represent a plurality of blood product constituents.
The donor stem cells or bone barrow are stored in sub-zero cryogenic temperatures in sterile non-breachable containers with means to preserve said stem cells or marrow indefinitely.
The collection is from harvesting stem cells collection means.
The means of collecting said stem cells is selected from an embryonic, umbilical, bone marrow, sterile mass blood, cellular dialysis, continuous circulating cell technique means and combination.
The collection of said stem cells is by means specific for harvesting targeted cells from the vascular compartment.
The collection of said stem cells is by concentration means specific for harvesting targeted cells from bone marrow.
The concentration of said stem cells, is by means sufficient to result in a plurality of stem cells, which are in combination with blood product constituents.
The supply of said stem cells collected and transfused is sufficient to achieve periodic global integration of biologically younger stem cells into donor-recipient's bone marrow & tissues throughout his/her body.
The collection obtains at least on the order of 10.sup.6 total nucleated cells per Kg. weight of the person in a single collection session.
The unit comprises a dosage of a fraction of the total nucleated cells required to be transfused or infused.
The units collected or transfused contain equal or different dosages.
The preserving or storage step is independent of tissue or HLA typing of the collected stem cells prior to storing in a stem cell bank.
The preserving or storage step comprises the step of determining from the collected stem cells at least a distinctive property associated with the person prior to storing in a the stem cell bank, so as to provide a means of secured identification to match the collected stem cells with the person at the time of use.
The typing step includes providing indicia with each unit representing information of said at least one distinctive property.
The indicia is embodied in at least one of a label, bar code, magnetic strip, and microchip.
The stem cells are collected during a pre-disease stage in which the person may be diagnosed with a health condition.
Securely enclosing said stem cells in non breachable containers suspended in bio-compatible nutrient media.
Labeling, dating & entering into a secure data base (with redundancy and system controls for accuracy and backup in alternate locations)
Storing said containers under liquid nitrogen.
The storing said stem cells is within a bio-compatible media suspension.
The periodic infusion results in integration of biologically younger stem cells into recipient tissues throughout his/her body at least once every five years, which are comprised of the recipient current chronologically older aged DNA-containing cells.
A composition containing DNA in a stem cell carrier and optionally bone marrow, whereby said DNA is delivered in sufficient useable quantity to an autologous donor/recipient to regenerate existing cell structure on a global body basis.
The composition of claim of 127, wherein said regeneration provides a 5 year differential between the donor/recipient's chronological age and his biological age.
The composition of claim of 127, wherein the age difference is no less than 20 years.
The composition of claim 127, wherein concentrated stem cells represent a plurality to a majority of the composition.
The activity resulting from said infusion into said recipient results in global cellar replacement of biologically younger DNA.
The collection methods of the aforementioned claims where at least 3 billion nucleated stem cells are collected per session.
The periodic collection and infusion methods of the aforementioned claims are pro-active, absent response or in anticipation of any disease.
The harvesting/collection of stem cells comprises making one or more insertions of a syringe means into the donor's hip or pelvic bone to extract bone marrow as said biological specimen containing stem cells.
The stem cells comprise adult hematopietic stem cells.
Introducing a stem cell growth stimulating agent into the donor prior to the harvesting in a manner effective to increase the population of the stem cells in the peripheral blood of the donor before harvesting the specimen.
Infusion constitutes routine cellular/tissue maintenance and administered prior to the onset of any disease, which may be contracted by recipient
An anti-aging blood serum composition comprising pluripotent stem cells, and normal blood constituents whereupon transfusion of said blood serum that recipient's blood contains 1.0% to 4.0.0% said stem cells compared to total nucleated cells contained in the blood stream, measured by the total number of CD+34 cells contained. in recipient's blood stream.
An anti-aging method comprising:
   periodic collection of stem cells from donor from birth to age 500 years
   sorting and concentrating said stem cells of step 1 in the collected sera contents, such that they represent a plurality of blood product constituents
   storing said donor-recipient stem cells of step 2 in sterile conditions in non-breachable containers under sub-zero temperature (equal or less than 0° F.)
   thawing a minor portion of said stored stem cells of step 3 after a period of 1 to 500 years
   periodic auto-transfusions of said thawed stem cells back into same donor (recipient) starting after said donor's chronological age 10,
   whereby said infusion results in an integration of biologically younger stem cells into recipient tissues throughout his/her body
The collection is from harvesting stem cells collection means.
The means is selected from a sterile mass blood collection, cellular dialysis, continuous circulating cell screening technique and combination.
The collection of said stem cells is by means specific for harvesting targeted cells from the vascular compartment
The collection of said stem cells is by concentration means specific for harvesting targeted cells from bone marrow.
The concentration of said stem cells, is by means sufficient to result in a plurality of stem cells, which are in combination with blood product constituents.
The collection results in storage of at least 50, 250, 500 or 1000 grams of stem cells or material containing a plurality to a majority of said stem cells
The mass is collected by donor's chronological age of 10, 20, 30, 40 or 50.
The periodic collection occurs one (1) to twelve (12) times annually.
The collection is no more frequently than every three months
The periodic collection is no more frequently than every 6 months
The collection of said stem cells is no less than every 5 years.
The collection is no less than once a year for a donor chronological age 1 & 2, no less than twice a year for a donor ages 3 to 5, three times a year ages 6 through 15, and annually for ages 16 years or older.
The a plurality to a majority of said collection occurs during the first 50 years of donor's chronological life.
The collection yield is increased through the use of agents selected from the group consisting of donor preparatory, promotion, induction, and circulation enhancers.
The agents increase the quantity, quality and accessibility of stem cells within the circulation.
After said concentration, said stem cells are pooled, packed and batched according to incremental age time frames of collection.
The incremental time frame is from birth to 2 years, 3 to 5 years, 5 to 10 years, 11 to 15 years, and in 5-year increments thereafter
The minimum mass of stem cells collected is equivalent to 10% to 100% of a standard unit of red blood cells (no less than 25 grams) standard in the blood banking industry.
The temperature is a cryogenic freezing temperature sufficient to insure long term storage of said stem cells.
The storage is under liquid nitrogen.
The periodic infusion results in integration of biologically younger stem cells into recipient tissues throughout his/her body, which are comprised of the recipient current chronologically older aged DNA-containing cells
The infusion results in the creation of a transient tissue chimera—age difference between said infused stem cells (collected and stored years earlier) and recipient's currently aged DNA containing cells is 20 years or greater.
Infusion incorporates harvested autologous stem cells, which have been collected at least 5, 10, 20, 30 or 40 years earlier
The infusion contains a mixture of stem cells collected over various dates
The infusion incorporates means for selecting stem cells for maximizing anti-aging response.
The infusion is by means of auto-transfusions (autologous infusions) of the owner donor-recipient of earlier collected stem cells
The infusion delivers said stem cells by means of indirect transportation via said donor's blood stream
The infusion is by single or multiple access port of entry into the blood stream, whereby said infused stem cells find their way to all body tissue sites
The stem cells, lodge and become integrated into said donor's cell structure (integrated component).
The integration is by self means
The infusion is characterized as being global, whereby autologous stem cells are delivered to all sites, throughout the recipient body, wherein
   biologically younger stem cells are integrated into recipient's tissues throughout his/her entire body
   a transient chimera is formed, characterized by the collective tissue composition of the recipient current chronologically older DNA-containing cells and biologically younger DNA-containing infused autologous stem cells, which were collected and stored years earlier
   the age differential of cells comprising the resulting transient tissue chimera is 5 years or greater
An anti-aging method comprising:
   Periodic collection of stem cells from a donor from birth to adult,
   Sorting and concentrating said stem cells in the collected sera contents, such that said stem cells represent a plurality of blood product constituents,
   storing said donor-recipient stem cells in sub-zero temperatures in sterile non-breachable containers as means to preserve said stem cells indefinitely,
   periodic and regular infusions of said stem cells into the donor starting after age 10 years, whereby,
   the continuation of said periodic collections and periodic infusions result in an average biological age of the new body comprised of replaced tissues from the donor DNA at least 5 years younger than the actual chronological age of said donor/recipient
The stem cell samples are collected within a collection period prior to donor's first infusion, whereby said stem cells have an average chronological age equivalent to their biological DNA age
The stem cell samples are collected within a collection period prior to donor's infusion, whereby upon infusion said stem cells have an average biological DNA age at least 5 years less than the chronological age of said donor/recipient
The difference between the infused biological DNA stem cell age and the chronological age of the donor/recipient increases, as the chronological age of the donor/recipient increases.
The difference between resulting average biological age of the recipient's transformed body following infusions of DNA-containing stem cells is at least 5, 10, 20, 30, 40, 50, 60 or 100 years younger than the recipient's chronological age
A composition whereby said DNA is delivered in sufficient useable quantity to an autologous donor/recipient to regenerate existing cell structure on a global body basis.
The composition wherein said regeneration provides a 5 year differential between the donor/recipient's chronological age and his biological age.
The composition wherein the age difference is no less than 20 years.
The composition wherein concentrated stem cells represent a plurality to a majority of the composition.
The activity resulting from said infusion into said donor results in global cellar replacement.

### Bone Marrow Methodologies

An in-vivo, optionally human, inchoate-transient tissue chimera structure comprised of tissue cells with at least two distinct DNA chronological ages [optionally with two distinct genomes], [optionally, whereby the age differential between DNA tissue age is at least 3 months, 6 months, 9 months, 1, 2, 3, 4, 5, 8, 10, 12, 15, 20, 30, 40, 60, 80, 90, or more, years].
The following methods and conditions are applicable in the alternative and/or selectively combined.
   The whole of the said transient chimera tissue structures is defined by the statistical preponderance of younger DNA containing cells incorporated throughout the majority of the recipient's body organs and tissues, thus establishing a ‘mature’ chimera.
   The younger tissues transmogrifies the recipient's initial older tissues.
   The transmogrification is result of a proactive periodic series of introductions/infusions containing SC and/or BM into a recipient's body structures and/orsera.
An ex-vivo bone marrow (BM) composition includes:
   a fraction of bone marrow selected from a matched donor, or recipient, and combination, and
   A fraction of stem cells (SC) selected from compatible or altered [NGvHR—non graft-versus-host rejecting, matched or HLA typing free] donor stem cells or recipient (autologous) stem cells, and combination,
      Optionally wherein said SCs comprise a plurality of primordial SCs selected from the group consisting of totipotent, pluripotent, omnipotent (multipotent) SCs, and combination.
      Optionally, wherein said plurality is at least or greater than 5%, of primordial stem cells.
      Optionally, being pluripotent and/or omnipotent (multipotent)
      Optionally, containing normal blood constituents or serum
      Optionally, wherein said SC are proliferated SCs
      Optionally, said SCs are derived from donor under the age of 30, optionally collected from donor blood, donor bone marrow, umbilical cord, placenta or undamaged embryo.
   Optionally, wherein said composition after the combination of both BM and SCs fractions becomes an activated composition, characterized as enjoying/generating greater anti-aging potency, regeneration capability and/or compatibility with recipient than prior to said combination.
An ex-vivo [recipient compatible or altered] bone marrow composition comprising a fraction of bone marrow selected from a matched donor and from recipient,
   Optionally, wherein recipients bone marrow is younger, and the age differential between donor's BM and recipient's BM is at least five years.
   Optionally, wherein said age differential between 3rd party donor and recipient is as of date of infusion a product containing said BM into recipient.
   Optionally wherein the ratio of the fraction of Donors BM to recipient's BM is more than 2:1
   Optionally, wherein said formulation/ratio enhances DNA compatibility with recipient
   Optionally, reducing/eliminating graft v host rejection
   Optionally, wherein said formulation/ratio enhances anti-aging potency.
   Optionally, wherein said donor BM is collected from recipient matched donor [optionally using HLA matching procedure]
   A fraction of stem cells (SC) selected from compatible or altered, NGvHR, matched or HLA typing free donor stem cells to recipient, recipient (autologous) stem cells, and combination,
      Optionally wherein said SCs comprise a plurality of primordial SCs selected from the group consisting of totipotent, pluripotent, omnipotent (multipotent) SCs, and combination.
      Optionally, wherein said SCs are proliferated SCs, optionally enhanced, augmented, proliferated
   Optionally, wherein said composition after the combination of both BM and SCs fractions becomes an activated composition, characterized as generating greater anti-aging potency, capability and/or compatibility than prior to said combination.
An ex-vivo [recipient compatible or altered] bone marrow composition comprising a bone marrow from a compatible or altered, NGvHR, or matched to recipient donor, wherein the age differential between donor's BM and recipient's chronological age is on average at least five years [or 50. 40, 30, 20, 10 years].
   Optionally wherein said BM is collected from compatible or altered donor, who is HLA type-matched or otherwise matched with recipient; or donated SCs are altered to be compatible or altered to recipient's DNA
   A fraction of stem cells (SC) selected from compatible or altered, NGvHR, matched, or HLA typing free donor stem cells, recipient (autologous) stem cells, and combination,
      Optionally wherein said SCs comprise a plurality of primordial SCs selected from the group consisting of totipotent, pluripotent, omnipotent (multipotent) SCs, and combination.
In vivo recipient compatible or altered BM composition comprising:
   Recipient's in-vivo BM,
   Optionally, compatible or altered 3^{rd} party donor BM
      Optionally, wherein said BM is donor collected from compatible or altered, NGvHR, or matched donor, [or Autologous/recipient Donor], and combination,
   Optionally, BM collected from autologous/recipient prior to [or approximately concurrent with] an infusion incorporating said recipient's BM.
   And, a fraction of stem cells selected from compatible or altered, NGvHR, matched, or HLA typing free donor SCs, or recipient (autologous) stem cells, and combination,
      Optionally, wherein said SC are proliferated ex-vivo SCs, and
      Optionally wherein a plurality of said SCs comprise totipotent, pluripotent, omnipotent SCs, and combination.
An recipient compatible or altered or NGvHR BM composition comprising:
   In-vivo recipient (autologous) bone marrow,
   Younger aged DNA containing Donor or Autologous SC's.
An ex-vivo [recipient own] bone marrow composition comprising:
   A fraction of bone marrow selected from a donor-recipient [autologous BM],
      Optionally, wherein donor-recipient bone marrow is chronologically younger than chronological age of recipient on date of an infusion by least five years]
   A fraction of stem cells (SC) selected from a 3^{rd} party donor compatible or altered, NGvHR, matched or HLA typing free donor stem cells, recipient (autologous) stem cells, and combination,
      Optionally wherein said SCs comprise a plurality of primordial SCs selected from the group consisting of totipotent, pluripotent, omnipotent SCs, and combination.
         Optionally, wherein said SC are proliferated SCs
An ex-vivo or in-vivo recipient compatible or altered composition comprising
   a fraction of bone marrow comprising donor and/or recipient BM, and
   a fraction of proliferated recipient's autologous SCs, matched, NGvHR, or HLA typing free 3^{rd} party donor SC, and combination, wherein said fraction contains at least a plurality of 5% primordial stem cells, selected from totipotent, pluripotent, omnipotent SCs, and combination.
A method of preparing an ex-vivo BM product, comprising:
   Collecting BM, from a 3rd party donor, or (autologous) recipient, and combination
      Optionally said BM is collected from matched donor;
      Optionally, wherein recipient's bone marrow is younger, and the age differential between donor's BM and recipient's BM is at least five years.
      Optionally, wherein said BM collected from autologous/recipient
      Optionally, wherein said BM is collected at a period prior to an infusion incorporating said BM, which is sufficient for its useful incorporation in this product.
      Optionally, said BM collected from recipient is at least 4-7 days, prior to date of an infusion incorporating said BM.
      Optionally, said BM collected from recipient is at least 1 years younger than chronological age of recipient.
      Optionally, said BM collected from recipient is prior to his age of 30 years or younger.
   Collecting SC from a matched 3^{rd} party donor, or source where SC's do not require HLA typing (embryonic, umbilical cord, placenta), or autologous source (recipient), and combination,
      Optionally, wherein said SC's are collected from an embryo (absent damage) or umbilical cord or placenta and combination, and/or 3^{rd} party donor age 10 years or younger than recipient.
      Optionally where said SC's are 3^{rd} party SCs at least 10 biological or chronological years younger than recipient
   Separating said stem cells into their primordial fraction, selected from the group consisting of totipotent, pluripotent and omnipotent SCs, and combination,
      optionally, selecting pluripotent and omnipotent SCs
      optionally pluripotent SCs only
      Optionally preparing a SC fraction wherein said SCs comprise a plurality of primordial SCs selected from the group consisting of totipotent, pluripotent, omnipotent SCs, and combination.
      Optionally, wherein said SC plurality is at least or greater than 5%, of the total of SCs contained in the fraction.
   Proliferating separated/collected primordial SCs
      Optionally proliferating totipotent, pluripotent, omnipotent SCs, only
   Combining a fraction of said proliferated SCs and BM together to create a product.
      Optionally, wherein said BM fraction is a combination of both donor and recipient BM and wherein said combination provides for a ratio of donor's BM fraction to fraction of recipient's BM greater than 2:1
         Optionally, a ratio wherein said formulation/ratio enhances DNA compatibility with recipient
         Optionally, a ratio wherein said formulation/ratio enhances anti-aging potency.
         Optionally in the activation step below.
      Optionally, wherein said BM fraction to SC fraction is greater than 2:1
         Optionally, a ratio wherein said formulation/ratio enhances DNA compatibility with recipient
         Optionally, a ratio wherein said formulation/ratio enhances anti-aging potency.
         Optionally in the activation step below.
      Optionally, activating, nurturing, stimulating and/or shocking [“Activating”] said product containing fractions of SCs and BM by exogenous and/or endogenous means (Activated Composition).
      Optionally, said activation is by exogenous means including:
      Signaling or stimulation via biological & chemical agents or means and physical agents or means, such as sound (harmonic resonance, ultra sonic or other), light or electronic wave (various spectrum including UV, radio, high to low wave length to infrared, etc.), electronic, magnetic, chemical, agitation/mixing, pressure changes, vacuum applications, temperature—thermo or cryo manipulations,
      other means (as provided in the referenced art), and combination
      Optionally, wherein said activation includes introduction of other inputs, including nutrients, preservation, augmentation ingredients or other means as provided in the referenced art:
         Providing matched or autologous blood cell and other components, constituents, minerals, elements, sera, protein and/or other product sufficient to nourish said composition
         Optionally, additionally providing normal blood cell and other constituents or sera.
      Optionally, wherein said activation occurs naturally as condition of combining BM and SC fraction
      Optionally, wherein said activation includes method to
      Preserve said BM and SC composition
      Enhance SC and/or BM growth and proliferation
      Inhibit or Enhance SC differentiation, if deemed desirable. Or other techniques such as may employ enhancement and augmentation of, through prior and/or ongoing exposure to inducing and/or stimulating biological, chemical, physical agents to include nanotechnology, radio, photonic, magnetic, electronic chemical, harmonic, thermo or cryogenic, hormonal agents and/or techniques and combinations thereof
      Optionally, wherein after the introduction of said primordial SC into the composition, and/or after said activation, said SC and/or BM [independently] proliferate and/or differentiate
         Optionally, wherein said SC proliferation or differentiation is independent of earlier SC proliferation [after collection prior to combination with BM].
         Optionally, wherein said activation proliferation is dependent upon method employed for earlier SC proliferation.
      Optionally, wherein said proliferation and/or differentiation of activation further results in stem cells selected from the group consisting of totipotent, pluripotent, omnipotent or progenitor SCs, and combination.
         Optionally, pluripotent SCs
      Optionally, wherein after activation said product increases in volume and/or potency.
         Optionally, wherein total SC population increases 2 to 6 times.
   Optionally, incubating/growing said activation product for a period of time,
      Optionally, said incubation period is sufficient to generate maximum potency, recipient compatibility, or product (SC and/or BM) volume, and combination.
      Optionally, said incubation is for 1 hour to 1 year.
         Optionally, at least 5 to 6 days [or other acceptable period ranging from 2 hours to 6 months, 1-6 hours, 4-6 hours, 2-24 hours, 1-5 days, 1-7 days, 3-8 days, 1-5 weeks, 4-6 weeks, 1-8 months, 4-6 months].
Optionally, processing, adopting, augmenting and/or preparing said product, as required, to become a compatible or altered infusion product for recipient, whereby infusion is made absent “graft v host” or immune sensitivity response
Optionally, wherein DNA attributes of recipient are conferred to the composition.
Optionally storing said product, any component or subcomponent of said product at any step or point prior to infusion.
   Storage of any component or subcomponent may optionally be at any step after collection of SC and/or bone marrow, prior to infusion/injection.
   Preparing a final composition/product suitable for infusion into recipient.

The following elements apply alternatively or selectively together to the above bone marrow methodologies
The composition contains bone marrow selected from both recipient and a compatible or altered or NGvHR donor.
A fraction of recipient bone marrow and a fraction of compatible or altered or NGvHR donor bone marrow.
An ex-vivo bone marrow composition includes altered or NGvHR donor bone marrow, recipient bone marrow, and a quantity of compatible or altered, NGvHR or HLA typing free donor primordial stem cells.
An ex-vivo bone marrow composition includes compatible or altered/matched or NGvHR donor bone marrow, recipient bone marrow and optionally a fraction of stem cells.
An ex-vivo bone marrow composition includes compatible or altered donor bone marrow of recipient, recipient bone marrow, and a quantity of compatible or altered pluripotent and/or omnipotent stem cells.
The bone marrow composition is augmented to become recipient compatible or altered or enhanced bone marrow.
The pluripotent and/or omnipotent stem cells are at least 10 years younger than recipient's chronological age, optionally being embroyonic, cord stem cells or placental SCs.
The bone marrow is harvested from a donor, optionally augmented and then expanded or proliferated
The volume of bone marrow to stem cells is greater than 1:1.
The volume of bone marrow to stem cells is at least 2:1
The volume of bone marrow to stem cells is at least 3:1
The combination has incubated for a period of at least 4 hours to 5 hours, days or weeks.
The composition becomes active after the combination of BM and SC fractions due to a change of temperature or other physical or biological factors or use of novel technologies to include nanotechnology and bio-engineering.
The composition becomes active after an endogenous stimulation.
After said primordial stem cells are introduced into a composition of proliferated SCs, said composition is activated, characterized by greater anti-aging potency than prior to said combination.
The activation is characterized by a volume increase in the total number of SCs.
An composition avoids “graft versus host” rejection or immune response of recipient.
The BM or SC are collected from compatible or altered, NGvHR, matched or HLA typed donors to recipient or other means suitable to insure compatibility to include altering the donated cellular DNA by various current and emerging technologies.
The 3^{rd} party donor BM and/or SCs are employed, where said BM and SCs are introduced/combined with recipient's BM and/or SCs under conditions and for a sufficient period of time to acquire recipient DNA characterization.
The bone marrow fraction is collected from a matched 3^{rd} party donor at least 10 years younger than recipient
The ex-vivo and in-vivo composition of claims, wherein said bone marrow fraction is collected from 3rd party donor, who is as of date of collection at least 30 years younger than recipient.
The ex-vivo composition of claims, wherein said BM fraction is solely or contains in part recipient's bone marrow
The ex-vivo composition of claims, wherein said BM fraction is solely or contains in part matching 3^{rd} party donor bone marrow.
The ex-vivo composition of claims, wherein said BM fraction has been augmented, refined, separated, and/or proliferated, in preparation for infusion into recipient and may employ selective inhibition methods and/or enhancements of—through prior and/or ongoing exposure to inducing and/or stimulating biological (including organisms—re-engineered retroviruses, other similar means), antibodies, immune reactants—stimulants, homeopathic chemicals, cellular growth or other hormonal agents; physical agents to include nanotechnology, radio, photonic, electronic, magnetic, chemical, harmonic, thermo or cryogenic and/or techniques, and combinations thereof.

The Appendix attached hereto includes definitions and other information applicable to and incorporated herein.

1. An autologous anti-aging method comprising:
cc. Periodic collection of stem cells and/or bone marrow from a donor from after conception to an adult chronological age,
dd. Providing for the long term storage of said donor-recipient stem cells and/or bone marrow in sterile conditions in containers,
ee. Thawing a portion of said stored stem cells and/or bone marrow after a period of storage, and
ff. Periodic and regular infusions or infusion of said stem cells and/or bone marrow into the donor starting after age 10 years, whereby said periodic infusions result in an average biological age of the new body comprised of replaced tissues from the donor DNA at least 5 years younger than the actual chronological age of said recipient.
